SEQUENCE LISTING (28 sequences)

SEQ ID NO: 1 - DNA, 22 bp, Artificial Sequence
  Synthesized
nngnnngnnnnnnnnngnnngn

SEQ ID NO: 2 - DNA, 22 bp, Artificial Sequence
  Synthesized
nngnnngnnnnnnnnngnnngn

SEQ ID NO: 3 - DNA, 32 bp, Artificial Sequence
  Synthesized
cctatgcgatcgagttttctngatngcatagg

SEQ ID NO: 4 - DNA, 32 bp, Artificial Sequence; identical to SEQ ID NO: 3
  Synthesized

SEQ ID NO: 5 - DNA, 32 bp, Artificial Sequence
  Synthesized
cctatgngatngagttttctngatngcatagg

SEQ ID NO: 6 - DNA, 32 bp, Artificial Sequence; identical to SEQ ID NO: 5
  Synthesized

SEQ ID NO: 7 - DNA, 16 bp, Artificial Sequence
  Synthesized
gtacatgngctccaga

SEQ ID NO: 8 - DNA, 16 bp, Artificial Sequence
  Synthesized
tctggagcgcatgtac

SEQ ID NO: 9 - DNA, 16 bp, Artificial Sequence
  Synthesized
gtacatgngctccaga

SEQ ID NO: 10 - DNA, 16 bp, Artificial Sequence
  Synthesized
tctggagngcatgtac

SEQ ID NO: 11 - DNA, 32 bp, Artificial Sequence; identical to SEQ ID NO: 3
  Synthesized

SEQ ID NO: 12 - DNA, 32 bp, Artificial Sequence; identical to SEQ ID NO: 5
  Synthesized

SEQ ID NO: 13 - DNA, 32 bp, Artificial Sequence
  Synthesized
cctatggcatgcagttttctgcatgccatagg

SEQ ID NO: 14 - DNA, 32 bp, Artificial Sequence; identical to SEQ ID NO: 5
  Synthesized

SEQ ID NO: 15 - DNA, 32 bp, Artificial Sequence
  Synthesized
cctatgngatngagttttctcgatcgcatagg

SEQ ID NO: 16 - DNA, 32 bp, Artificial Sequence; identical to SEQ ID NO: 15
  Synthesized

SEQ ID NO: 17 - DNA, 32 bp, Artificial Sequence; identical to SEQ ID NO: 5
  Synthesized

SEQ ID NO: 18 - DNA, 32 bp, Artificial Sequence; identical to SEQ ID NO: 5
  Synthesized

SEQ ID NO: 19 - DNA, 32 bp, Artificial Sequence; identical to SEQ ID NO: 15
  Synthesized

SEQ ID NO: 20 - DNA, 32 bp, Artificial Sequence; identical to SEQ ID NO: 5
  Synthesized

SEQ ID NO: 21 - DNA, 16 bp, Artificial Sequence
  Synthesized
gtacatgcgctccaga

SEQ ID NO: 22 - DNA, 16 bp, Artificial Sequence
  Synthesized
tctggagcgnatgtac

SEQ ID NO: 23 - DNA, 16 bp, Artificial Sequence
  Synthesized
gtacatgngctccaga

SEQ ID NO: 24 - DNA, 16 bp, Artificial Sequence
  Synthesized
tctggagngcatgtac

SEQ ID NO: 25 - DNA, 32 bp, Artificial Sequence; identical to SEQ ID NO: 5
  Synthesized

SEQ ID NO: 26 - DNA, 32 bp, Artificial Sequence; identical to SEQ ID NO: 5
  Synthesized

SEQ ID NO: 27 - DNA, 26 bp, Artificial Sequence
  Synthesized
nngnnngnnnnnnnnnnnnngnnngn

SEQ ID NO: 28 - DNA, 26 bp, Artificial Sequence
  Synthesized
nngnnngnnnnnnnnnnnnngnnngn

(19) United States
(12) Patent Application Publication
(10) Pub. No.: US 2015/0167004 A1
(43) Pub. Date: Jun. 18, 2015

(54) OLIGONUCLEOTIDE INHIBITORS OF DNA METHYLTRANSFERASES AND THEIR USE IN TREATING DISEASES

(71) Applicant: METHEOR THERAPEUTICS CORPORATION, Shoreline, WA (US)

(72) Inventors: Andrew Z. Sledziewski, Shoreline, WA (US); Theodore Devos, Seattle, WA (US); Ryszard Kole, Cambridge, MA (US)

(21) Appl. No.: 14/413,004

(22) PCT Filed: Jul. 9, 2013
(86) PCT No.: PCT/US13/49624

§ 371 Date: Jan. 5, 2015

Related U.S. Application Data
(60) Provisional application No. 61/669,606, filed on Jul. 9, 2012.

(51) Int. Cl. C12N 15/113

(52) U.S. Cl. CPC C12N 15/1137; C12N 2310/3341; C12N 2310/531

(57) ABSTRACT

Modified oligonucleotides comprising CpG sites, wherein the cytosine is replaced by cytosine analogs are provided as well as methods of making the oligonucleotides and their use in inhibiting DNA Methyltransferase, inhibiting or reversing methylation of genes and in treating cancer, tumorigenesis and hyper-proliferative disorders.

### CROSS-REFERENCE TO RELATED APPLICATIONS

This application claims the benefit of U.S. Provisional Application No. 61/669,606, filed Jul. 9, 2012, the disclosure of which is hereby incorporated by reference.

### TECHNICAL FIELD

The invention relates generally to design, synthesis, production and application of oligonucleotide analogues and to methods of using such oligonucleotide analogues for treatment of disease, including cancer, tumor, and angiogenesis in mammals, including humans and animals. In particular, the invention relates to methods of using oligonucleotides containing cytosine analogues as therapeutics for hypo-methylating aberrantly methylated genes in human cancer leading to restoration of aberrantly methylated gene expression. The present invention relates to design, synthesis and application of novel oligonucleotides containing cytosine analogues for use in modifying DNA methylation, and which are useful as therapeutics. Oligonucleotide analogues are provided that incorporate various analogues of cytosine in the oligonucleotide sequence, including, but not limited to 5-aza-cytidine, 5-aza-2′-deoxycytidine, 5-fluoro-cytidine, fluorocyclopentenylcytosine, zebularine, or deoxyzebularine. Such oligonucleotide analogues can be used as hypomethylating agents for modulation of DNA methylation, especially for effective inhibition of methylation of cytosine at the C-5 position. Methods for synthesizing these oligonucleotide analogues and for modulating C-5 cytosine methylation are provided.

### BACKGROUND OF THE INVENTION

Two hypomethylating agents Vidaza® (5-aza-cytidine) and Dacogen® (5-aza-2′-deoxycytidine) are currently being used as new pharmaceuticals for the treatment of chronic myelogenous leukemia (CML), myelodysplastic syndrome (MDS), non-small cell lung (NSCL) cancer, sickle-cell anemia, and acute myelogenous leukemia (AML). One of the functions of these agents is their ability to inhibit DNA methylation. DNA methylation is an epigenetic effect common to many systems. This modification involves the covalent modification of cytosine at the C-5 position. In higher eukaryotes, portions of genomic DNA are often methylated at cytosines followed by guanosine in CpG dinucleotides. This modification has important regulatory effects on gene expression, especially when involving CpGs located in the promoter regions of many genes. Aberrant methylation of normally un-methylated CpG-containing promoters has been shown to affect transcriptional activity of the downstream genes. In many cancers aberrant methylation leads to transcriptional inactivation of defined tumor suppressor genes. Therefore, restoring transcriptional activity of tumor suppressor genes by hypomethylating drugs can lead to a powerful new form of anti-tumor therapies.

Both drugs, 5-aza-cytidine and 5-aza-2′-deoxycytidine need to be converted into their active forms; in case of 5-aza-2′-deoxycytidine the phosphorylated 5-aza-deoxycytidine, in case of 5-aza-cytidine conversion to deoxyribose form and phosphorylation. After conversion to their triphosphate form by deoxycytidine kinase, both compounds are incorporated into replicating DNA at a rate similar to that of the natural substrate, dCTP (Bouchard and Momparler 1983 Mol. Pharmacol. 24:109-114). After chromosomal duplication, in order to conserve existing methylation pattern, the 5-methylcytosine on the parental strand serves as a guide to DNA methyltransferases to direct methylation of the complementary daughter DNA strand. The replacement of cytosine with hypomethylating analogues at CpG sites produces an irreversible inactivation of DNA methyltransferases by covalently trapping the enzyme by hypomethylating analogues in the DNA (Juttermann et al. 1994 Proc. Natl. Acad. Sci. USA 91:11797-11801). This unique mechanism of action of existing hypomethylating agents allows genes silenced (that were once methylated) from previous rounds of cell division to be re-expressed. After further DNA synthesis and cell cycle division, progeny strands from the hemi-methylated DNA result in DNA strands that are completely un-methylated at these sites (Jones P. 2001 Nature 409: 141, 143-4). By specifically inhibiting DNA methyltransferases aberrant methylation of the tumor suppressor genes could be reversed.

Despite its proven antileukemic effects in CML, MDS, and AML, the potential application of hypomethylating agents have been hampered by delayed and prolonged myelosuppression. Lower doses of both Vidaza® and Dacogen®, given over a longer period of time, have minimized myelosuppression to manageable levels without compromising its ability to suppress cancer via its hypo-methylation effect. At higher doses, the associated toxicity was prohibitive. However, treatment of hematologic and solid tumors at maximally tolerated doses of hypomethylating agents has been ineffective. The cause of myelosuppression is not clear. It is plausible that since hypomethylating agents are randomly and extensively incorporated into the DNA, including bone marrow cells that are involved in normal hematopoiesis, the severe DNA damage due to the instability of Vidaza® and Dacogen® leads to necrosis. Since incorporation of hypomethylating agents is not restricted to only the CpG-rich sequences, the DNA can break, due to the instability of the agents, and require repair at numerous sites outside of the CpG islands.

Vidaza® and Dacogen® are unstable in aqueous media and undergo hydrolytic degradation. In acidic medium, Dacogen® is hydrolyzed at room temperature to 5-aza-cytosineazacytosine and 2-deoxyribose. In neutral medium at room temperature, the opening of the triazine ring takes place at the 6-position to form the transient intermediate formyl derivative, which further degrades to the amidino-urea derivative and formic acid (Piskala, A.; Synackova, M.; Tomankova, H.; Fiedler, P.; Zizkowsky, V. Nucleic Acids Res. 1978, 4, s109-s-113.). This hydrolysis at the 6-position occurs in acidic and basic aqueous media at even faster rates.

In view of the chemical instability and toxicities associated with Vidaza® and Dacogen®, there exists a need to develop more stable and superior hypomethylating agents, that are not specific to a gene sequence and are not incorporated into the genomic DNA and that provide effective hypo-methylation without significantly affecting the integrity of the DNA or causing gene toxicity.

The recitation of any reference in this application is not an admission that the reference is prior art to this application.

### SUMMARY OF THE INVENTION

The invention will best be understood by reference to the following detailed description of the aspects and embodiments of the invention, taken in conjunction with the accompanying drawings. The discussion below is descriptive, illustrative and exemplary and is not to be taken as limiting the scope defined by any appended claims.

The present invention provides oligonucleotide inhibitors of DNA methyltransferases (DNMT) and their use in treating diseases. Each oligonucleotide inhibitor contains at least one modified CpG dinucleotide target sequence for DNMT, in which the CpG is modified by replacing the cytosine (C) in one strand by a cytosine analogue and the C in the opposite strand is either unmodified or it is replaced by methylated cytosine (such as 5-methylcytosine) to create a hemi-methylated target for DNMT. Oligonucleotide inhibitors described herein contain at least one CpG site that incorporate analogues of the cytosine nucleotide in at least one CpG dinucleotide present in such oligonucleotide. In some instances, the oligonucleotide inhibitors contain at least 2, at least 3, at least 4, at least 5, and at least 6 CpG sites, in which some or up to all of the CpG sites incorporate an analogue of cytosine or a 5-methylcytosine substitution of cytosine.

Cytosine nucleotide analogues include but are not limited to 5-aza-cytidine, 5-aza-2′-deoxycytidine, 5-fluoro-cytidine, fluorocyclopentenylcytosine, zebularine, or deoxyzebularine. These oligonucleotides are configured as either a self-complementary single stranded sequence that forms a stem-loop structure, or as complementary oligonucleotides that form a double stranded sequence when annealed.

In one aspect, the invention provides an isolated or synthetic oligonucleotide comprising the nucleotide sequence of SEQ ID NO: 1 or SEQ ID NO: 2. In an embodiment, the oligonucleotide forms a stem-loop (hairpin) structure. In a further embodiment, nucleotides in positions 10-13 or in positions 15-18 form the loop structure. In a further embodiment, the length of the oligonucleotide is at least sufficient to form the stem-loop (hairpin) structure at human physiological temperature, or human body temperature. In further embodiments, the oligo nucleotide occurs in the stem-loop (hairpin) structure at a temperature in the range of 35.5° C. to 39° C., or the temperature is 35.5° C., 36° C., 36.5° C., 37° C., 37.5° C., 38° C., 38.5° C., 39° C., or 39.5° C.

In another aspect, the invention provides and isolated or synthetic oligonucleotide comprising or consisting of a sequence of nucleotides having at least 50% sequence identity to the nucleotide sequence of SEQ ID NO: 3, to the nucleotide sequence of SEQ ID NO: 4, to the nucleotide sequence of SEQ ID NO: 5, to the nucleotide sequence of SEQ ID NO: 6, to the nucleotide sequence of SEQ ID NO: 7 to the nucleotide sequence of SEQ ID NO: 8, to the nucleotide sequence of SEQ ID NO: 9, to the nucleotide sequence of SEQ ID NO: 10, to the nucleotide sequence of SEQ ID NO: 11, to the nucleotide sequence of SEQ ID NO: 12, to the nucleotide sequence of SEQ ID NO: 13, to the nucleotide sequence of SEQ ID NO: 14, to the nucleotide sequence of SEQ ID NO: 15, to the nucleotide sequence of SEQ ID NO: 16, to the nucleotide sequence of SEQ ID NO: 17, to the nucleotide sequence of SEQ ID NO: 18, to the nucleotide sequence of SEQ ID NO: 19, to the nucleotide sequence of SEQ ID NO: 20, to the nucleotide sequence of SEQ ID NO: 21, to the nucleotide sequence of SEQ ID NO: 22, to the nucleotide sequence of SEQ ID NO: 23, to the nucleotide sequence of SEQ ID NO: 24, to the nucleotide sequence of SEQ ID NO: 25, or to the nucleotide sequence of SEQ ID NO: 26. In an embodiment, the oligonucleotide forms a stem-loop (hairpin) structure. In an embodiment, the isolated or synthetic oligonucleotide is not incorporated into the genome and is not specific to any gene sequence. In a further embodiment, the nucleotides in positions 10-13 or in positions 15-18 form the loop structure. In another embodiment, the loop structure consists of 3 nucleotides. In further embodiments, the sequence of nucleotides has at least 55%, 60%, 65%, 60%, 75%, 80%, 85%, 90%, or 95% sequence identity to the nucleotide sequence, or at least 96%, 97%, 98%, or 99% sequence identity to the nucleotide sequence. In further embodiments the oligonucleotide is between 22 and 50 nucleotides, is at least 33, at least 40, or at least 50 nucleotides, is at least 23, 24, or 25 nucleotides, is at least 26, 27, 28, 29, or 30 nucleotides, is at least 31, 32, 33, 34, or 35 nucleotides, is at least 36, 37, 38, 39, or nucleotides, is at least 40, 41, 42, 43, 44, or 45 nucleotides, or is at least 46, 47, 48, 49, or 50 nucleotides. In another embodiment, the isolated or synthetic oligonucleotide further comprises a 5′ or a 3′ extension of up to 5, 6, 7, 8 9, or 10 nucleotides. In a further embodiment, the oligonucleotide comprises a 5′ and a 3′ extension.

In another aspect, the invention provide an isolated or synthetic oligonucleotide consisting of the nucleotide sequence of SEQ ID NO: 1, an isolated or synthetic oligonucleotide consisting of the nucleotide sequence of SEQ ID NO: 2, an isolated or synthetic oligonucleotide consisting of the nucleotide sequence of SEQ ID NO: 3, an isolated or synthetic oligonucleotide consisting of the nucleotide sequence of SEQ ID NO: 4, an isolated or synthetic oligonucleotide consisting of the nucleotide sequence of SEQ ID NO: 5, an isolated or synthetic oligonucleotide consisting of the nucleotide sequence of SEQ ID NO: 6, an isolated or synthetic oligonucleotide consisting of the nucleotide sequence of SEQ ID NO: 7, an isolated or synthetic oligonucleotide consisting of the nucleotide sequence of SEQ ID NO: 8, an isolated or synthetic oligonucleotide consisting of the nucleotide sequence of SEQ ID NO: 9, an isolated or synthetic oligonucleotide consisting of the nucleotide sequence of SEQ ID NO: 10, an isolated or synthetic oligonucleotide consisting of the nucleotide sequence of SEQ ID NO: 11, or an isolated or synthetic oligonucleotide consisting of the nucleotide sequence of SEQ ID NO: 12, an isolated or synthetic oligonucleotide consisting of the nucleotide sequence of SEQ ID NO: 13, an isolated or synthetic oligonucleotide consisting of the nucleotide sequence of SEQ ID NO: 14, an isolated or synthetic oligonucleotide consisting of the nucleotide sequence of SEQ ID NO: 15, an isolated or synthetic oligonucleotide consisting of the nucleotide sequence of SEQ ID NO: 16, an isolated or synthetic oligonucleotide consisting of the nucleotide sequence of SEQ ID NO: 17, or an isolated or synthetic oligonucleotide consisting of the nucleotide sequence of SEQ ID NO: 18, an isolated or synthetic oligonucleotide consisting of the nucleotide sequence of SEQ ID NO: 19, an isolated or synthetic oligonucleotide consisting of the nucleotide sequence of SEQ ID NO: 20, an isolated or synthetic oligonucleotide consisting of the nucleotide sequence of SEQ ID NO: 21, an isolated or synthetic oligonucleotide consisting of the nucleotide sequence of SEQ ID NO: 22, an isolated or synthetic oligonucleotide consisting of the nucleotide sequence of SEQ ID NO: 23, or an isolated or synthetic oligonucleotide consisting of the nucleotide sequence of SEQ ID NO: 24, an isolated or synthetic oligonucleotide consisting of the nucleotide sequence of SEQ ID NO: 25, or an isolated or synthetic oligonucleotide consisting of the nucleotide sequence of SEQ ID NO: 26. In an embodiment, the isolated or synthetic oligonucleotide is not incorporated into the genome and is not specific to any gene sequence.

In another aspect the invention provides an isolated or synthetic oligonucleotide comprising or consisting of a sequence of nucleotides, wherein the sequence comprises a 5′ extension and a structure-loop (hairpin), wherein the structure-loop (hairpin) comprises or consists of the sequence of nucleotides selected from the group consisting of: a nucleotide sequence of SEQ ID NO: 1; a nucleotide sequence of SEQ ID NO: 2; a nucleotide sequence of SEQ ID NO: 3; a nucleotide sequence of SEQ ID NO: 4; a nucleotide sequence of SEQ ID NO: 5; and a nucleotide sequence of SEQ ID NO: 6. In an embodiment, the sequence comprises a 3′ extension of up to 5, 6, 7, 8, 9, or 10 nucleotides. a nucleotide sequence of SEQ ID NO: 6, a nucleotide sequence of SEQ ID NO: 7; a nucleotide sequence of SEQ ID NO: 8; a nucleotide sequence of SEQ ID NO: 9; a nucleotide sequence of SEQ ID NO: 10; a nucleotide sequence of SEQ ID NO: 11; a nucleotide sequence of SEQ ID NO: 12, a nucleotide sequence of SEQ ID NO: 13; a nucleotide sequence of SEQ ID NO: 14; a nucleotide sequence of SEQ ID NO: 15; a nucleotide sequence of SEQ ID NO: 16; a nucleotide sequence of SEQ ID NO: 17; a nucleotide sequence of SEQ ID NO: 18, a nucleotide sequence of SEQ ID NO: 19; a nucleotide sequence of SEQ ID NO: 20; a nucleotide sequence of SEQ ID NO: 21; a nucleotide sequence of SEQ ID NO: 22; a nucleotide sequence of SEQ ID NO: 23; a nucleotide sequence of SEQ ID NO: 24, a nucleotide sequence of SEQ ID NO: 25; and a nucleotide sequence of SEQ ID NO: 26. In an embodiment, the sequence comprises a 3′ extension of up to 5, 6, 7, 8, 9, or 10 nucleotides. In an embodiment, the isolated or synthetic oligonucleotide is not incorporated into the genome and is not specific to any gene sequence.

In another aspect, the invention includes an isolated or synthetic oligonucleotide comprising a sequence of nucleotides, wherein the sequence comprises a structure-loop (hairpin) and a 3′ extension wherein the structure-loop (hairpin) portion comprises or consists of the sequence of nucleotides selected from the group consisting of: a nucleotide sequence of SEQ ID NO: 1; a nucleotide sequence of SEQ ID NO: 2; a nucleotide sequence of SEQ ID NO: 3; a nucleotide sequence of SEQ ID NO: 4; a nucleotide sequence of SEQ ID NO: 5; and a nucleotide sequence of SEQ ID NO: 6. In an embodiment, the sequence comprises a 5′extension of up to 5, 6, 7, 8, 9, or 10 nucleotides. a nucleotide sequence of SEQ ID NO: 6, a nucleotide sequence of SEQ ID NO: 7; a nucleotide sequence of SEQ ID NO: 8; a nucleotide sequence of SEQ ID NO: 9; a nucleotide sequence of SEQ ID NO: 10; a nucleotide sequence of SEQ ID NO: 11; a nucleotide sequence of SEQ ID NO: 12, a nucleotide sequence of SEQ ID NO: 13; a nucleotide sequence of SEQ ID NO: 14; a nucleotide sequence of SEQ ID NO: 15; a nucleotide sequence of SEQ ID NO: 16; a nucleotide sequence of SEQ ID NO: 17; a nucleotide sequence of SEQ ID NO: 18, a nucleotide sequence of SEQ ID NO: 19; a nucleotide sequence of SEQ ID NO: 20; a nucleotide sequence of SEQ ID NO: 21; a nucleotide sequence of SEQ ID NO: 22; a nucleotide sequence of SEQ ID NO: 23; a nucleotide sequence of SEQ ID NO: 24, a nucleotide sequence of SEQ ID NO: 25; and a nucleotide sequence of SEQ ID NO: 26. In an embodiment, the sequence comprises 5′ extension of up to 5, 6, 7, 8, 9, or 10 nucleotides. In an embodiment, the isolated or synthetic oligonucleotide is not incorporated into the genome and is not specific to any gene sequence.

In another aspect, the invention includes, an isolated or synthetic oligonucleotide comprising the following linked components: a first sequence of nucleotide or nucleotides; a first cytosine residue or cytosine analogue residue, wherein the cytosine analogue residue is 5-fluoro-cytidine, fluorocyclopentenylcytosine, zebularine or deoxyzebularine; a first guanine residue; a second sequence of nucleotide or nucleotides; a third sequence of nucleotide or nucleotides; a fourth sequence of nucleotide or nucleotides; a second cytosine residue or cytosine analogue residue, wherein the cytosine analogue residue is 5-fluoro-cytidine, fluorocyclopentenylcytosine, or zebularine, or deoxyzebularine; a second guanine residue; and a fifth sequence of nucleotide or nucleotides; wherein the second and fourth sequences of nucleotides are complementary to each other forming a hairpin loop of the third sequence of nucleotides, and wherein the nucleotides of in the first, second, third, fourth, and fifth sequences comprise adenine, cytosine, guanine, and thymine. In an embodiment, the first and fifth sequences of nucleotides are complementary to each other. In another embodiment, the third sequence of nucleotides comprises 3, 4, 5, 6 or 7 nucleotides. In another embodiment, the oligonucleotide comprises a sequence selected from the group consisting of GENERAL FORMULA A, B, C, and D. In another embodiment, the oligonucleotide comprises the sequence of SEQ ID NO: 1 or the sequence of SEQ ID NO: 2. In another embodiment, the oligonucleotide nucleotide sequence comprises at least 11 nucleotides. In another embodiment, the nucleotide comprises 12, 13, 14, or 15 nucleotides. In another embodiment the nucleotide sequence comprises at least 16 nucleotides. In another embodiment, the nucleotide comprises 17, 18, 19, or 20 nucleotides. In another embodiment, the nucleotide sequence comprises at least 21 nucleotides. In another embodiment, the nucleotide comprises 22, 23, 24, or 25 nucleotides. In another embodiment, the nucleotide sequence comprises at least 26 nucleotides. In another embodiment, the nucleotide comprises 27, 28, 29, or 30 nucleotides. In another embodiment, the nucleotide sequence comprises at least 31 nucleotides. In another embodiment, the nucleotide comprises at least 32 nucleotides. In another embodiment, the nucleotide sequence comprises between 11 and 32 nucleotides. In another embodiment, the nucleotide sequence comprises at least 33, at least 40, or at least 50 nucleotides.

The invention also provides, an isolated or synthetic oligonucleotide comprising at least 11 nucleotides in length and at least one CpG site, wherein the 5′ sequence of the oligonucleotide is complementary to the 3′ sequence of the oligonucleotide and forms a double-stranded DNA complex with it forming a hairpin loop structure, wherein the CpG site is located in the 5′ or 3′ sequences, or in both the 5′ and the 3′ forming the stem sequence, and wherein the cytosine of the CpG site is an cytosine analogue selected from the group consisting of 5-fluoro-cytidine, fluorocyclopentenylcytosine, zebularine and deoxyzebularine. In another embodiment, the CpG site is located in the 5′ sequence. In another embodiment, the CpG site is located in the 3′ sequence. In another embodiment, the cytosine of the sequence that is complementary to the guanine of the CpG site is 5-methyl-cytosine. In another embodiment, the oligonucleotide is annealed to it complementary sequence. In another embodiment, the 5′ end of the oligonucleotide is annealed to the 3′ end of the oligonucleotide forming the stem of the stem-loop structure.

In another aspect, the invention includes an isolated or synthetic oligonucleotide comprising the sequence of SEQ ID NO: 7 or SEQ ID NO: 8, wherein the oligonucleotide is annealed to it complementary sequence. In another embodiment, the oligonucleotide sequence is at least 6 nucleotides in length. In another embodiment, the complementary sequence is at least 6 nucleotides in length. In another embodiment, the oligonucleotide sequence and the complementary sequences are each at least 6 nucleotides in length. In another embodiment, the sequence of the oligonucleotide comprises at least one CpG site. In another embodiment, the complementary sequence comprises at least one CpG site. In another embodiment, the cytosine of the CpG site is a cytosine analogue selected from the group consisting of 5-aza-cytidine, 5-aza-2′-deoxycytidine, 5-fluoro-cytidine, fluorocyclopentenylcytosine, zebularine, and deoxyzebularine. In another embodiment, the cytosine of the CpG site of the complementary sequence is 5-methyl-cytosine. In an embodiment, the isolated or synthetic oligonucleotide is not incorporated into the genome and is not specific to any gene sequence.

In another aspect, the invention provides an isolated or synthetic pair of oligonucleotides comprising the nucleotide sequence of SEQ ID NO: 7 and its complementary sequence. In another aspect, the invention provides an isolated or synthetic pair of oligonucleotides comprising the nucleotide sequence of SEQ ID NO: 8 and its complementary sequence. In another embodiment, the oligonucleotide sequence is at least 6 nucleotides in length. In another embodiment, the complementary sequence is at least 6 nucleotides in length. In another embodiment, the oligonucleotide sequence and the complementary sequences are each at least 6 nucleotides in length. In another embodiment, the sequence of the oligonucleotide comprises at least one CpG site. In another embodiment, the complementary sequence comprises at least one CpG site. In another embodiment, the cytosine of the CpG site is a cytosine analogue selected from the group consisting of 5-aza-cytidine, 5-aza-2′-deoxycytidine, 5-fluoro-cytidine, fluorocyclopentenylcytosine, zebularine and deoxyzebularine. In another embodiment, the cytosine of the CpG site of the complementary sequence is 5-methyl-cytosine. In an embodiment, the isolated or synthetic oligonucleotide is not incorporated into the genome and is not specific to any gene sequence.

In another aspect, the invention provides a pharmaceutical composition comprising the isolated or synthetic oligonucleotide or pair of oligonucleotides according to any previous claim or their pharmaceutically acceptable salt or ester and a pharmaceutically-acceptable carrier. In another embodiment, a salt or ester of the isolated or synthetic oligonucleotide or pair of oligonucleotides is provided. In another embodiment, the backbone of the isolated or synthetic oligonucleotide or pair of oligonucleotides comprises either a phosphodiester linker or artificial backbone. In an embodiment, the backbone comprises internucleoside linkages including, without limitation, phosphodiester, phosphorothioate, phosphorodiamidate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages.

In another aspect, the invention provides a composition comprising an agent selected from the group consisting of: a) an isolated or synthetic oligonucleotide comprising or consisting of the nucleotide sequence of SEQ ID NO:1; b) an isolated or synthetic oligonucleotide comprising or consisting of the nucleotide sequence of SEQ ID NO:2; c) an isolated or synthetic oligonucleotide comprising or consisting of the nucleotide sequence of SEQ ID NO:3; d) an isolated or synthetic oligonucleotide comprising or consisting of the nucleotide sequence of SEQ ID NO:4; e) an isolated or synthetic oligonucleotide comprising or consisting of the nucleotide sequence of SEQ ID NO:5; f) an isolated or synthetic oligonucleotide comprising or consisting of the nucleotide sequence of SEQ ID NO:6; g) an isolated or synthetic oligonucleotide comprising or consisting of the nucleotide sequence of SEQ ID NO:7; h) an isolated or synthetic oligonucleotide comprising or consisting of the nucleotide sequence of SEQ ID NO:8; i) an isolated or synthetic oligonucleotide comprising or consisting of the nucleotide sequence of SEQ ID NO:9; j) an isolated or synthetic oligonucleotide comprising or consisting of the nucleotide sequence of SEQ ID NO:10; k) an isolated or synthetic oligonucleotide comprising or consisting of the nucleotide sequence of SEQ ID NO:11; l) an isolated or synthetic oligonucleotide comprising or consisting of the nucleotide sequence of SEQ ID NO:12; m) an isolated or synthetic oligonucleotide comprising or consisting of the nucleotide sequence of SEQ ID NO:13; n) an isolated or synthetic oligonucleotide comprising or consisting of the nucleotide sequence of SEQ ID NO:14; o) an isolated or synthetic oligonucleotide comprising or consisting of the nucleotide sequence of SEQ ID NO:15; q) an isolated or synthetic oligonucleotide comprising or consisting of the nucleotide sequence of SEQ ID NO:16; r) an isolated or synthetic oligonucleotide comprising or consisting of the nucleotide sequence of SEQ ID NO:17; s) an isolated or synthetic oligonucleotide comprising or consisting of the nucleotide sequence of SEQ ID NO:18; t) an isolated or synthetic oligonucleotide comprising or consisting of the nucleotide sequence of SEQ ID NO:19; u) an isolated or synthetic oligonucleotide comprising or consisting of the nucleotide sequence of SEQ ID NO:20; v) an isolated or synthetic oligonucleotide comprising or consisting of the nucleotide sequence of SEQ ID NO:21; w) an isolated or synthetic oligonucleotide comprising or consisting of the nucleotide sequence of SEQ ID NO:22; x) an isolated or synthetic oligonucleotide comprising or consisting of the nucleotide sequence of SEQ ID NO:23; y) an isolated or synthetic oligonucleotide comprising or consisting of the nucleotide sequence of SEQ ID NO:24; z) an isolated or synthetic oligonucleotide comprising or consisting of the nucleotide sequence of SEQ ID NO:25; aa) an isolated or synthetic oligonucleotide comprising or consisting of the nucleotide sequence of SEQ ID NO:26; bb) a an isolated or synthetic oligonucleotide comprising or consisting of the nucleotide sequence of GENERAL FORMULA A; cc) an isolated or synthetic oligonucleotide comprising or consisting of the nucleotide sequence of GENERAL FORMULA B; dd) an isolated or synthetic oligonucleotide comprising or consisting of the nucleotide sequence of GENERAL FORMULA C; ee) an isolated or synthetic oligonucleotide comprising or consisting of the nucleotide sequence of GENERAL FORMULA D; ff) an isolated or synthetic oligonucleotide comprising or consisting of the nucleotide sequence of GENERAL FORMULA E, gg) an isolated or synthetic oligonucleotide comprising or consisting of the nucleotide sequence of GENERAL FORMULA F; hh); an isolated or synthetic pair of oligonucleotides comprising or consisting of the nucleotide sequence of SEQ ID NO:7, and its complementary sequence; ii) an isolated or synthetic pair of oligonucleotides comprising or consisting of the nucleotide sequence of SEQ ID NO:8, and its complementary sequence; jj) an isolated or synthetic pair of oligonucleotides comprising or consisting of the nucleotide sequence of SEQ ID NO:7 and the nucleotide sequence of SEQ ID NO:8; ll) an isolated or synthetic pair of oligonucleotides comprising or consisting of the nucleotide sequence of GENERAL FORMULA F, and its complementary sequence; and any combination of two or more of a)-ll), or a subset thereof. In an embodiment, the isolated or synthetic oligonucleotide is not incorporated into the genome and is not specific to any gene sequence.

In another aspect, the invention provides a composition comprising an agent selected from the group consisting of: a) an isolated or synthetic oligonucleotide comprising or consisting of the nucleotide sequence MTC-422. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-423. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-425. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-427. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-432. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-433. In an embodiment, the oligonucleotide comprises an artificial backbone. In another embodiment, the oligonucleotide comprises a phosphorothioate backbone. In an embodiment, the oligonucleotide comprises a phosphodiester back bone.

In another aspect, the invention provides a composition comprising an agent selected from the group consisting of: a) an isolated or synthetic oligonucleotide comprising or consisting of the nucleotide sequence MTC-434. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-424F. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-424R. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-429F. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence MTC-429R. In an embodiment, the oligonucleotide comprises a phosphodiester backbone. In another embodiment, the oligonucleotide comprises an artificial backbone. In another embodiment, the oligonucleotide comprises a phosphorothioate backbone.

In another aspect, the invention provides a composition comprising an agent selected from the group consisting of: a) an isolated or synthetic oligonucleotide comprising or consisting of the nucleotide sequence selected from the group consisting of: MTC-422N, MTC-423N, MTC-425N, MTC-427N, MTC-432N, MTC-433N, MTC-424FN, MTC-424RN, MTC-429FN, and MTC-429RN. In an embodiment, the oligonucleotide comprises a phosphodiester backbone. In another embodiment, the oligonucleotide comprises an artificial backbone. In another embodiment, the oligonucleotide comprises a phosphorothioate backbone.

The invention provides a method for reducing, limiting, inhibiting, or minimizing the amount of DNMT in a cell comprising contacting the cell under suitable conditions with an agent comprises the composition(s) described herein, or a pharmaceutically acceptable salt or ester thereof, and whereby the DNMT is reduced, limited, inhibited, or minimized. In another embodiment, DNMT1 is reduced preferentially to DNMT3a and DNMT3b. In another embodiment, DNMT3a is reduced preferentially to DNMT1 and DNMT3b. In another embodiment, DNMT3b is reduced preferentially to DNMT1 and DNMT3a. In another embodiment, activity of the DNMT in the cell is eliminated.

In another aspect, the invention provides an isolated cell having a reduced amount of DNMT from the methods described herein.

The invention further provides a method for reducing, limiting, inhibiting, or minimizing methylation of a cell, comprising contacting the cell under suitable conditions with an agent that comprises a composition described herein, or a pharmaceutically acceptable salt or ester thereof, and whereby methylation in the cell is reduced, limited, inhibited, or minimized. In another embodiment, the invention provides an isolated cell having a reduced amount of methylation from the method.

In an aspect, the invention provides a method for reverting aberrant methylation of a cell, comprising contacting the cell under suitable conditions with an agent that comprises a composition described herein, or a pharmaceutically acceptable salt or ester thereof, and whereby aberrant methylation in the cell is reverted in whole or in part.

The invention provides a method for restoring hypo-methylation of a tumor suppressor gene, comprising contacting a cell under suitable conditions with an agent that comprises a composition described herein, or a pharmaceutically acceptable salt or ester thereof, and whereby the tumor suppressor gene is hypo-methylated in whole or in part.

The invention also provides a method for restoring transcriptional activity of tumor suppressor genes by contacting a cell under suitable conditions with an hypomethylating agent that comprises a composition described herein, or a pharmaceutically acceptable salt or ester thereof, and whereby transcriptional activity of tumor suppressor genes is restored whole or in part.

In another aspect, the invention provides a method of introducing re-expression of one or more methylation-silenced tumor suppressor genes by contacting a cell under suitable conditions with an hypomethylating agent that comprises a composition described herein, or a pharmaceutically acceptable salt or ester thereof, and whereby re-expression of methylation-silenced tumor suppressor genes is restored whole or in part.

The invention also provides a method of inhibiting, reducing, limiting, or minimizing tumorgenecity of a gene by contacting a cell under suitable conditions with an hypomethylating agent that comprises a composition described herein, or a pharmaceutically acceptable salt or ester thereof, and whereby tumorgenecity of the gene is inhibited, reduced, limited, or minimized in whole or in part.

In another aspect, the invention provides a method of treating a DNMT-related disease or disorder in a subject in need thereof, the method comprising administering to the subject a therapeutically effective amount of a composition described herein. In an embodiment, the DNMT-related disease or disorder is a cell proliferative disorder. In a further embodiment, the cell proliferative disorder is selected from the group consisting of acute-myeloid leukemia (AML), chronic myeloid leukemia (CML), or myelodysplastic syndromes (MDS), cancers of the liver or kidney, a liver proliferative disorder or a kidney disorder, cancers of the lung or a lung proliferative disorder or a kidney disorder, cancers of the ovaries or an ovarian proliferative disorder, as well as breast cancer, colorectal cancer and pancreatic cancer.

In another aspect, the invention provides methods for treating acute-myeloid leukemia (AML), chronic myeloid leukemia (CML), or myelodysplastic syndromes (MDS), or a liver proliferative disorder, a kidney disorder, a lung or ovary, breast, colorectal or pancreatic cancer, prolerativ disorder or disease with the isolated or synthetic oligonucleotides of SEQ ID NO: 1, SEQ ID NO:2, SEQ ID NO:3, SEQ ID NO: 4, SEQ ID NO: 5, or SEQ ID NO: 6, the oligonucleotide pairs of SEQ ID NO: 7 and SEQ ID NO: 8, the oligonucleotide pairs of SEQ ID NO: 9 and SEQ ID NO: 10, SEQ ID NO: 11, SEQ ID NO:12, SEQ ID NO:13, SEQ ID NO: 14, SEQ ID NO: 15, or SEQ ID NO: 16 SEQ ID NO: 17, SEQ ID NO:18, SEQ ID NO:19, SEQ ID NO: 20, SEQ ID NO: 21, or SEQ ID NO: 22, SEQ ID NO: 23, SEQ ID NO:24, SEQ ID NO:25, or SEQ ID NO: 26. Such treatment includes administration of any of the oligonucleotides alone or in combination.

In one aspect the invention provides a general formula for the stem loop oligonucleotide of: 5′ R[N₁]XGR[N₂]B[N]R[N_{2c}]XGR[N_{1c}] 3′, referred to herein as “GENERAL FORMULA A”, where N represents the number of nucleotides; R represents any of four nucleotides (adenine, cytosine, guanine and thymine); B represents any of four nucleotides (adenine, cytosine, guanine and thymine) forming the loop structure; X represents cytosine or a cytosine analogue (analogues include but are not limited to 5-aza-cytidine, 5-aza-2′-deoxycytidine, 5-fluoro-cytidine, fluorocyclopentenylcytosine or zebularine, or deoxyzebularine); G represents Guanine; the 5′ end of the oligonucleotide is complementary to the 3′ end; and wherein R[N₁] nucleotides are complementary to R[N_{1c}] nucleotides, R[N₂] nucleotides are complementary to R[N_{2c}] nucleotides, and B[N] nucleotides are not complementary to each other and are preferably all the same nucleotide, more preferably thymine. In an embodiment, when X is a cytosine analogue, the C of the CpG site opposite the analog is 5-methylcytosine.

In one aspect the invention provides a general formula for the stem loop oligonucleotide of: 5′R[N₁]XGR[N₂]B[N]R[N_{2c}]XGR[N_{1c}] 3′, referred to herein as “GENERAL FORMULA B”, where N represents the number of nucleotides; R represents any of four nucleotides (adenine, cytosine, guanine and thymine); B represents any of four nucleotides (adenine, cytosine, guanine and thymine) forming the loop structure; X represents cytosine or a cytosine analogue (analogues include but are not limited to 5-fluoro-cytidine, fluorocyclopentenylcytosine or zebularine, or deoxyzebularine); G represents Guanine; the 5′ end of the oligonucleotide is complementary to the 3′ end, and wherein R[N₁] nucleotides are complementary to R[N_{1c}] nucleotides, R[N₂] nucleotides are complementary to R[N_{2c}] nucleotides, and B[N] nucleotides are not complementary to each other and are preferably all the same nucleotide, more preferably thymine. In an embodiment, when X is a cytosine analogue, the C of the CpG site opposite the analog is 5-methylcytosine.

In one aspect the invention provides a general formula for the stem loop oligonucleotide of: 5′R[N₁]XGR[N₂]XGR[N₃]B[N]R[N_{3c}]XGR[N_{2c}]XGR[N_{1c}]3′, referred to herein as “GENERAL FORMULA C”, where N represents the number of nucleotides; R represents any of four nucleotides (adenine, cytosine, guanine and thymine); B represents any of four nucleotides (adenine, cytosine, guanine and thymine) forming the loop structure; X represents cytosine or a cytosine analogue (analogues include but are not limited to 5-aza-cytidine, 5-aza-2′-deoxycytidine, 5-fluoro-cytidine, fluorocyclopentenylcytosine or zebularine, or deoxyzebularine); G represents Guanine; the 5′ end of the oligonucleotide is complementary to the 3′ end; and wherein R[N₁] nucleotides are complementary to [N_{1c}] nucleotides, R[N₂] nucleotides are complementary to R[N_{2c}] nucleotides, R[N₃] nucleotides are complementary to R[N_{3c}] nucleotides, and B[N] nucleotides are not complementary to each other and are preferably all the same nucleotide, more preferably thymine. In an embodiment, when X is a cytosine analogue, the C of the CpG site opposite the analog is 5-methylcytosine.

In one aspect the invention provides a general formula for the stem loop oligonucleotide of: 5′ R[N₁]XGR[N₂]XGR[N₃]B[N]R[N_{3c}]XGR[N₂]XGR[N_{1c}], referred to herein as “GENERAL FORMULA D”, where N represents the number of nucleotides; R represents any of four nucleotides (adenine, cytosine, guanine and thymine); B represents any of four nucleotides (adenine, cytosine, guanine and thymine) forming the loop structure; X represents cytosine or a cytosine analogue (analogues include but are not limited to 5-fluoro-cytidine, fluorocyclopentenylcytosine or zebularine, or deoxyzebularine); G represents Guanine; the 5′ end of the oligonucleotide is complementary to the 3′ end; and wherein R[N₁] nucleotides are complementary to R[N_{1c}] nucleotides, R[N₂] nucleotides are complementary to R[N_{2c}] nucleotides, R[N₃] nucleotides are complementary to R[N_{3c}] nucleotides, and B[N] nucleotides are not complementary to each other and are preferably all the same nucleotide, more preferably thymine. In an embodiment, when X is a cytosine analogue, the C of the CpG site opposite the analog is 5-methylcytosine.

In another aspect, the invention provides a general formula for the complementary oligonucleotide compounds that anneal to form a double stranded compound is: 5′ B[_{N}]XGB[_{N}] 3′, referred to herein as “GENERAL FORMULA E”, and it's complementary sequence, where B represents a string of N number of any of four nucleotides (adenine, cytosine, guanine and thymine), X represents cytosine or a cytosine analogue (analogues include but are not limited to 5-aza-cytidine, 5-aza-2′-deoxycytidine, 5-fluoro-cytidine, fluorocyclopentenylcytosine or zebularine, or deoxyzebularine) and G represents Guanine.

In one aspect the invention provides a general formula for the stem loop oligonucleotide of: 5′R[N₁]XGR[N₂]XGR[N₃]B[N]R[N_{3c}]XGR[N_{2c}]XGR[N_{1c}] 3′, referred to herein as “GENERAL FORMULA F”, where N represents the number of nucleotides; R represents any of four nucleotides (adenine, cytosine, guanine and thymine); B represents any of four nucleotides (adenine, cytosine, guanine and thymine) forming the loop structure; X represents cytosine or a cytosine analogue (analogues include but are not limited to 5-fluoro-cytidine, fluorocyclopentenylcytosine or zebularine, or deoxyzebularine); G represents Guanine; the 5′ end of the oligonucleotide is complementary to the 3′ end; and wherein R[N₁] nucleotides are complementary to R[N_{1c}] nucleotides, R[N₂] nucleotides are complementary to R[N_{2c}] nucleotides, R[N₃] nucleotides are complementary to R[N_{3c}] nucleotides, and B[N] nucleotides are not complementary to each other and are preferably all the same nucleotide, more preferably thymine. In an embodiment, when X is a cytosine analogue, the C of the CpG site opposite the analog is 5-methylcytosine.

In one aspect of the invention, an isolated or synthetic oligonucleotide analogue containing at least one CpG site and having 11 or more bases in length is provided. The 5′ and 3′ ends of the oligonucleotide have 3 or more complementary bases, such that in certain conditions they can form a double-strand to make a stem loop-shaped structure (hairpin). In one embodiment, the oligonucleotide is in the form of a stable hairpin structure at 36 degrees C. or higher. In an embodiment, the oligonucleotides of the present invention comprise at least one cytosine analogue selected from the group consisting of 5-aza-cytidine, 5-aza-2′-deoxycytidine, 5-fluoro-cytidine, fluorocyclopentenylcytosine, zebularine, or deoxyzebularine as a base residue replacing a cytosine in a CpG dinucleotide located in the stem of the hairpin. In an embodiment, the oligonucleotide can have more than one CpG dinucleotide and comprises more than one cytosine analog as described herein or a combination of two or more of said analogs. In another embodiment, the cytosine of the sequence that is complementary to the guanine of the CpG site is 5-methyl-cytosine. In another embodiment, the oligonucleotide is annealed to it complementary sequence.

In one another aspect of the invention, an isolated or synthetic oligonucleotide analogue containing at least one CpG site and having 11 or more bases in length is provided, adopting a hairpin conformation at 36 degrees C. and having at least one cytosine analogue selected from 5-aza-cytidine, 5-aza-2′-deoxycytidine, 5-fluoro-cytidine, fluorocyclopentenylcytosine, zebularine, or deoxyzebularine as a base residue replacing a cytosine in a CpG dinucleotide located in the stem of the hairpin and paired with a modified CpG dinucleotide where the cytosine has been replaced by 5-methyl-cytosine.

In another aspect of the invention, an isolated or synthetic double stranded oligonucleotide analogue containing at least one CpG site and having 6 or more bases in length is provided, has at least one cytosine analogue selected from 5-aza-cytidine, 5-aza-2′-deoxycytidine, 5-fluoro-cytidine, fluorocyclopentenylcytosine, zebularine, or deoxyzebularine as a base residue replacing a cytosine in a CpG dinucleotide and maintaining double stranded conformation at 36 degrees C.

In another aspect of the invention, an isolated or synthetic double stranded oligonucleotide analogue containing at least one CpG site and having 6 or more bases in length is provided, and that has at least one cytosine analogue selected from 5-aza-cytidine, 5-aza-2′-deoxycytidine, 5-fluoro-cytidine, fluorocyclopentenylcytosine, zebularine, or deoxyzebularine as a base residue replacing a cytosine in a CpG dinucleotide, paired with a modified CpG dinucleotide where the cytosine has been replaced by 5-methyl-cytosine and maintaining double stranded conformation at 36 degrees C.

The present invention also provides methods for synthesizing the modified oligonucleotides and methods for reducing, minimizing, inhibiting, or reversing aberrant DNA methylation in various disease conditions. Also provided are various building blocks for synthesizing the modified oligonucleotides, formulating and administering these modified oligonucleotides or compositions to treat conditions, such as cancer and hematological disorders.

In one or more aspects, the oligonucleotides containing fluoro-cytidine, fluorocyclopentenylcytosine, zebularine, or deoxyzebularine and derivatives, are provided. Also provided are methods for preparing, formulating and administering these compounds or compositions as therapeutics to a host in need thereof. In addition, the invention includes use of such cytosine analogue containing oligonucleotides for treatment of human or animal disease, including but not limited to cancer, tumor, and angiogenesis. The cytosine analogue containing oligonucleotides can be used as a medicament or in the manufacture of a medicament for treating cancer, tumor, and angiogenesis. The cytosine analogue containing oligonucleotides can be used for treatment of cancer, tumor, and angiogenesis.

In one aspect the sequences of the invention are shown in Table A, below, as well as those described herein.

TABLE A
Sequences

| SEQ ID NO: | sequence | position description |
|---|---|---|
| SEQ ID NO:1 | nnGnnnGnnnnnnnnnGnnnGn | Positions 1, 4, 5 ,8, 9, 10, 11, 12, 13, 14, |
| | | 15, 18, 19, and 22: n = A, C, G, or T |
| | | Positions 2, 6: n = Cytosine or 5- |
| | | methylcytosine |
| | | Positions 16, 20: n = 5-aza-cytidine, 5-aza- |
| | | 2′-deoxycytidine, 5-fluoro-cytidine, |
| | | fluorocyclopentenylcytosine, zebularine, or |
| | | deoxyzebularine |
| | | Position 1 is complementary to Position 22; |
| | | Position 4 is complementary to Position 19; |
| | | Position 5 is complementary to Position 18; |
| | | Position 8 is complementary to Position 15; |
| | | Position 9 is complementary to Position 14; |
| | | and |
| | | Positions 10-13 form the loop structure and |
| | | are identical to each other and are |
| | | preferably t |
| SEQ ID NO:2 | nnGnnnGnnnnnnnnnGnnnGn | Positions 1, 4, 5, 8, 9, 10, 11, 12, 13, 14, |
| | | 15, 18, 19, and 22: n = A, C, G, or T |
| | | Positions 2, 6: n = 5-aza-cytidine, 5-aza-2′- |
| | | deoxycytidine, 5-fluoro-cytidine, |
| | | fluorocyclopentenylcytosine, zebularine, or |
| | | deoxyzebularine |
| | | Positions 16, 20: n = Cytosine or 5- |
| | | methylcytosine |
| | | Position 1 is complementary to Position 22; |
| | | Position 4 is complementary to Position 19; |
| | | Position 5 is complementary to Position 18; |
| | | Position 8 is complementary to Position 15; |
| | | Position 9 is complementary to Position 14; |
| | | and |
| | | Positions 10-13 form the loop structure and |
| | | are identical to each other and are |
| | | preferably t |
| SEQ ID NO: 3 | cctatgcgatcgagttttctngatngcatagg | Positions 21 and 25: n = 5-aza-5,6-dihydro- |
| MTC-422 | | dC |
| Oligonucleotide | | |
| SEQ ID NO: 4 | cctatgcgatcgagttttctngatngcatagg | Positions 21 and 25: n = zebularine |
| MTC-423 | | (pyrimidin-2-one ribonucleoside) |
| Oligonucleotide | | |
| SEQ ID NO: 5 | cctatgngatngagttttctngatngcatagg | Positions 7 and 11: n = 5-methylcytosine |
| MTC-422m | | Positions 21 and 25: n = 5-aza-5,6-dihydro- |
| (also called | | dC |
| MTC-425) | | |
| Oligonucleotide | | |
| SEQ ID NO: 6 | cctatgngatngagttttctngatngcatagg | Positions 7 and 11: n = 5-methylcytosine |
| MTC-423m | | Positions 21 and 25: n = zebularine |
| (also called | | (pyrimidin-2-one ribonucleoside) |
| MTC-427) | | |
| Oligonucleotide | | |
| SEQ ID NO: 7 | gtacatgngctccaga | Position 8: n = 5-aza-5,6-dihydro-dC |
| MTC-424F | | |
| Oligonucleotide | | |
| SEQ ID NO:8 | tctggagcgcatgtac | none |
| MTC-424R | | |
| Oligonucleotide | | |
| SEQ ID NO: 9 | gtacatgngctccaga | Position 8: n = 5-aza-5,6-dihydro-dC |
| MTC-429F | | |
| SEQ ID NO: 10 | tctggagngcatgtac | Position 8: n = 5-methylcytosine |
| MTC-429R | | |
| SEQ ID NO: 11 | cctatgcgatcgagttttctngatngcatagg | Positions 21 and 25: n = deoxy-zebularine |
| MTC-432 | | |
| SEQ ID NO: 12 | cctatgngatngagttttctngatngcatagg | Positions 7 and 11: n = 5-methylcytosine; |
| MTC-433 | | Positions 21 and 25: n == deoxy-zebularine |
| SEQ ID NO: 13 | cctatggcatgcagttttctgcatgccatagg | none |
| MTC-434 | | |
| SEQ ID NO: 14 | cctatgngatngagttttctngatngcatagg | Positions 7, 11: n = 5-methylcytosine; |
| MTC-443 | | Positions 21, 25: n = deoxy-zebularine |
| SEQ ID NO: 15 | cctatgngatngagttttctcgatcgcatagg | Positions 7 and 11: n = 5-aza-5,6-dihydro- |
| MTC-422N | | dC |
| SEQ ID NO: 16 | cctatgngatngagttttctcgatcgcatagg | Positions 7 and 11: n = zebularine |
| MTC-423N | | (pyrimidin-2-one ribonucleoside) |
| SEQ ID NO: 17 | cctatgngatngagttttctngatngcatagg | Positions 7 and 11: n = 5-aza-5,6-dihydro- |
| MTC-425N | | dC; |
| | | Positions 21 and 25: n = 5-methylcytosine |
| SEQ ID NO: 18 | cctatgngatngagttttctngatngcatagg | Positions 7 and 11: n = zebularine |
| MTC-427N | | (pyrimidin-2-one ribonucleoside) |
| | | Positions 21 and 25: n = 5-methylcytosine |
| SEQ ID NO: 19 | cctatgngatngagttttctcgatcgcatagg | Positions 7 and 11: n = deoxy-zebularine; |
| | | MTC-432N |
| SEQ ID NO: 20 | cctatgngatngagttttctngatngcatagg | Positions 7 and 11: n = deoxy-zebularine; |
| MTC-433N | | Positions 21 and 25: n = 5-methylcytosine |
| SEQ ID NO: 21 | gtacatgcgctccaga | |
| MTC-424FN | | |
| SEQ ID NO: 22 | tctggagcgnatgtac | Position 10: n = 5-aza-5,6-dihydro-dC |
| MTC-424RN | | |
| SEQ ID NO: 23 | gtacatgngctccaga | Position 8: n = 5-methylcytosine |
| MTC-429FN | | |
| SEQ ID NO: 24 | tctggagngcatgtac | Position 8: n = 5-aza-5,6-dihydro-dC |
| MTC-429RN | | |
| SEQ ID NO: 25 | cctatgngatngagttttctngatngcatagg | Positions 7 and 11: n = cytosine or 5- |
| MTC-Con1 | | methylcytosine; |
| | | Positions 21 and 25: n = 5-aza-5,6-dihydro- |
| | | dC; 5-Aza-2 deoxycytidine; zebularine; or |
| | | deoxyzebularine |
| SEQ ID NO: 26 | cctatgngatngagttttctngatngcatagg | Positions 7 and 11: n = 5-aza-5,6-dihydro- |
| MTC-Con2 | | dC; 5-Aza-2 deoxycytidine; zebularine; or |
| | | deoxyzebularine; |
| | | Positions 21 and 25: n = cytosine or 5- |
| | | methylcytosine |
| SEQ ID NO: 27 | nnGnnnGnnnnnnnnnnnnnGnnnGn | Position 1: n = A, C, G, or T and is |
| | | complementary to Position 26, |
| | | Position 2: n = Cytosine or 5- |
| | | methylcytosine |
| | | Position 4: n = A, C, G, or T and is |
| | | complementary to Position 23; |
| | | Position 5: n = A, C, G, or T and is |
| | | complementary to Position 22; |
| | | Position 6: n = Cytosine or 5- |
| | | methylcytosine |
| | | Position 8: n = A, C, G, or T and is |
| | | complementary to Position 19; |
| | | Position 9: n = A, C, G, or T and is |
| | | complementary to Position 18; |
| | | Position 10: n = A, C, G, or T and is |
| | | complementary to Position 17; |
| | | Position 11: n = A, C, G, or T and is |
| | | complementary to Position 16; |
| | | Positions 12-15 form the loop structure and |
| | | are not complementary to each other, are |
| | | identical to each other: n = A, C, G, or T |
| | | and are preferably T |
| | | Positions 20 and 24: n = 5-aza-cytidine, 5- |
| | | aza-2′-deoxycytidine, 5-fluoro-cytidine, |
| | | fluorocyclopentenylcytosine, zebularine, or |
| | | deoxyzebularine |
| SEQ ID NO: 28 | nnGnnnGnnnnnnnnnnnnnGnnnGn | Position 1: n = A, C, G, or T and is |
| | | complementary to Position 26, |
| | | Position 2: n = 5-aza-cytidine, 5-aza-2′- |
| | | deoxycytidine, 5-fluoro-cytidine, |
| | | fluorocyclopentenylcytosine, zebularine, or |
| | | deoxyzebularine |
| | | Position 4: n = A, C, G, or T and is |
| | | complementary to Position 23; |
| | | Position 5: n = A, C, G, or T and is |
| | | complementary to Position 22; |
| | | Position 6: n = 5-aza-cytidine, 5-aza-2′- |
| | | deoxycytidine, 5-fluoro-cytidine, |
| | | fluorocyclopentenylcytosine, zebularine, or |
| | | deoxyzebularine |
| | | Position 8: n = A, C, G, or T and is |
| | | complementary to Position 19; |
| | | Position 9: n = A, C, G, or T and is |
| | | complementary to Position 18; |
| | | Position 10: n = A, C, G, or T and is |
| | | complementary to Position 17; |
| | | Position 11: n = A, C, G, or T and is |
| | | complementary to Position 16; |
| | | Positions 12-15 form the loop structure and |
| | | are not complementary, are identical to |
| | | each other: n = A, C, G, or T and are |
| | | preferably T |
| | | Positions 20 and 24: n = Cytosine or 5- |
| | | methylcytosine |

In another aspect is provided a method for reducing, limiting, inhibiting, or minimizing methylation of a cell, comprising contacting the cell under suitable conditions with an agent that comprises a composition comprising or consisting of the oligonucleotide of GENERAL FORMULA A, B, C, D, E, or F, or a pharmaceutically acceptable salt or ester thereof, whereby methylation in the cell is reduced, limited, inhibited, or minimized, and whereby the oligonucleotide is not incorporated into the genome and is not specific to any gene sequence. In a further aspect is provided an isolated cell having a reduced amount of methylation from the method.

In another aspect the invention provides a method for reverting aberrant methylation of a cell, comprising contacting the cell under suitable conditions with an agent that a composition comprising or consisting of the oligonucleotide of GENERAL FORMULA A, B, C, D, E, or F, or a pharmaceutically acceptable salt or ester thereof, whereby aberrant methylation in the cell is reverted in whole or in part, and whereby the oligonucleotide is not incorporated into the genome and is not specific to any gene sequence.

In another aspect the invention provides a method for restoring hypo-methylation of a tumor suppressor gene, comprising contacting a cell under suitable conditions with an agent that comprises a composition comprising or consisting of the oligonucleotide of GENERAL FORMULA A, B, C, D, E, or F, or a pharmaceutically acceptable salt or ester thereof, whereby the tumor suppressor gene is hypo-methylated in whole or in part, and whereby the oligonucleotide is not incorporated into the genome and is not specific to any gene sequence.

In another aspect the invention provides a method for restoring transcriptional activity of a tumor suppressor gene by contacting a cell under suitable conditions with an hypomethylating agent that comprises a composition comprising or consisting of the oligonucleotide of GENERAL FORMULA A, B, C, D, E, or F, or a pharmaceutically acceptable salt or ester thereof, whereby transcriptional activity of tumor suppressor genes is restored whole or in part, and whereby the oligonucleotide is not incorporated into the genome and is not specific to any gene sequence.

In another aspect the invention provides a method of introducing re-expression of a methylation-silenced tumor suppressor gene by contacting a cell under suitable conditions with an hypomethylating agent that comprises a composition comprising or consisting of the oligonucleotide of GENERAL FORMULA A, B, C, D, E, or F, or a pharmaceutically acceptable salt or ester thereof, whereby re-expression of one or more methylation-silenced tumor suppressor genes is restored in whole or in part, and whereby the oligonucleotide is not incorporated into the genome and is not specific to any gene sequence.

In another aspect the invention provides a method of inhibiting, reducing, limiting, or minimizing tumorgenecity of a gene by contacting a cell under suitable conditions with an hypomethylating agent that comprises a composition comprising or consisting of the oligonucleotide of GENERAL FORMULA A, B, C, D, E, or F, or a pharmaceutically acceptable salt or ester thereof, whereby tumorgenecity of the gene is inhibited, reduced, limited, or minimized in whole or in part, and whereby the oligonucleotide is not incorporated into the genome and is not specific to any gene sequence. In an embodiment, the cell proliferative disorder is selected from the group consisting of acute-myeloid leukemia (AML), chronic myeloid leukemia (CML), or myelodysplastic syndromes (MDS), cancers of the liver or kidney, or a liver proliferative disorder or a kidney disorder, cancer of the ovaries or an ovarian proliferative disorder, as well as cancers or cell-proliferative disorders of breast, colorectal or pancreatic cancers.

In another aspect the invention provides use of the oligonucleotide DNMT inhibitor in combination with other methylation inhibiting or gene demethylating drugs and other cell-killing drugs, such as chemotherapeutics. Other methylation inhibiting drugs include, but are not limited to, histone deacetylase inhibitors, such as hydroxamic acid derivatives, to enhance reversal of epigenetic silencing. See, for example, Cameron E E, Bachman K E, Myohanen S, Herman J G, Baylin S B. Synergy of Demethylation and Histone Deacetylase Inhibition in the Re-expression of Genes Silenced in Cancer. Nat Genet 1999; 21:103-7. The isolated or synthetic oligonucleotides described herein can be administered concurrently or simultaneously with the histone deacetylase inhibitors.

In addition, the oligonucleotide inhibitors described herein can be used with cytotoxic drugs, such as one or more chemotherapeutic drugs. In this sense, resistance of tumors to treatment with cisplatin, carboplatin, temozolomide, sorafenib (Nexavar®), and epirubicin, and the like can be decreased with the addition of, or in combination therapy with, the isolated or synthetic oligonucleotides of the present invention. In this way, the isolated or synthetic oligonucleotides described herein can be administered concurrently or simultaneously with the cytotoxic drugs. For example, the isolated or synthetic oligonucleotide DNMT inhibitors described herein can be given in advance of the cytotoxic drug, providing a period of demethylation as a window of epigenetic sensitization for the combination therapy. Moreover, demethylation may sensitize tumors to existing cytostatic therapies or make cells more receptive for epigenetic reprogramming. Similarly, the isolated or synthetic oligonucleotide inhibitors described herein may be used to sensitize tumors by epigenetic reactivation of pro-apoptotic genes or tumor suppressor genes that potentiate the effects of the cytotoxic drugs. See, for example, Plumb J A, Strathdee G, Sludden J, Kaye S B, Brown R. Reversal of Drug Resistance in Human Tumor Xenografts by 2′-deoxy-5-azacytidine-induced Demethylation of the hMLH1 Gene Promoter. Cancer Res 2000; 60:6039-44; and Soengas M S, Capodieci P, Polsky D, Mora J, Esteller M, Opitz-Araya X, et al. Inactivation of the Apoptosis Effector Apaf-1 in Malignant Melanoma. Nature 2001; 409:207-11. In an embodiment, the isolated and synthetic oligonucleotide DNMT inhibitors described herein can be used in place of, in combination with, in conjunction with, or as a supplement to chemotherapies, such as in place of, in combination with, in conjunction with, or as a supplement to sorafenib (Nexavar®) for liver cancer, including sorafenib-resistant liver cancer. In an embodiment, the isolated and synthetic oligonucleotide DNMT inhibitors described herein can be used in place of, in combination with, in conjunction with, or as a supplement to cisplatin for ovarian cancer, including cisplatin resistant ovarian cancer. Additionally, chemotherapeutic resistant cancers of the breast, lung, colorectal and pancreas can be similarly treated.

### BRIEF DESCRIPTION OF THE FIGURES

FIG. 1 shows the inhibition of the enzyme DNA methyltransferase 1 (DNMT1) with illustrative hypomethylating oligonucleotides, MTC-422, MTC-423, and MTC-424.

FIG. 2 shows the inhibition of the enzyme DNA methyltransferase 3a (DNMT3a) with illustrative hypomethylating oligonucleotides, MTC-422, MTC-423, and MTC-424.

FIG. 3 shows the inhibition of the enzyme DNA methyltransferase 3b1 (DNMT3b1) with illustrative hypomethylating oligonucleotides, MTC-422, MTC-423, and MTC-424.

FIG. 4 shows the chemical structure of the cytosine analog, 5-fluorocytidine.

FIG. 5 shows the chemical structure of the cytosine analog, 5-aza-cytidine.

FIG. 6 shows the chemical structure of the cytosine analog, 5-aza-2′-deoxycytidine.

FIG. 7 shows the chemical structure of the cytosine analog, Zebularine.

FIG. 8 shows the chemical structure of the cytosine analog, Deoxy-Zebularine.

FIG. 9 shows the results of MTC-433 compound re-activating a INK4A tumor suppressor gene.

FIG. 10 shows a comparison of MTC-433, MTC-427, and MTC-422 in DNMT1 biochemical inhibition assay.

### DESCRIPTION OF THE INVENTION

Unless stated otherwise, the following terms and phrases as used herein are intended to have the following meanings:

The term “oligonucleotide” refers to a polynucleotide formed from a plurality of linked nucleotide units, which may include, for example, deoxyribonucleotides or ribonucleotides, synthetic, natural, non-natural, engineered, or modified nucleotides; phosphodiester or modified linkages; synthetic, natural, non-natural, engineered, or modified bases; natural, non-natural sugars or modified sugars; nucleotide analogs, or combinations of these components. The nucleoside units may be part of viruses, bacteria, cell debris or oligonucleotide-based compositions (for example, siRNA and microRNA). Such oligonucleotides can also be obtained from existing nucleic acid sources, including genomic or cDNA, or can be produced by synthetic methods. The nucleoside residues can be coupled to each other by any of the numerous internucleoside linkages. Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone intemucleoside linkages. As used herein, the term “oligonucleotide” is not limited to a nucleotide sequence of a particular length.

“Compound”, as in the terms “compound of the formula”, “compound of the structure”, “compound of the invention”, and the like, shall refer to and encompass the chemical compound itself as well as, whether explicitly stated or not, and unless the context makes clear that the following are to be excluded: amorphous and crystalline forms of the compound, including polymorphic forms, where these forms may be part of a mixture or in isolation; free acid and free base forms of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in admixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and solvates, however, this reference is for emphasis only, and is not to be construed as excluding other of the above forms as identified above.

The term “operably linked”, when referring to nucleotide segments, indicates that the segments are arranged so that they function in concert for their intended purposes.

The term “pharmaceutically acceptable salts” or “pharmaceutically acceptable esters” refers to physiologically and pharmaceutically acceptable salts or esters of the compounds of the invention: i.e., or esters salts that retain the desired biological activity of the parent compound and do not impart undesired toxicological effects thereto.

When trade names are used herein, applicants intend to independently include the trade name product formulation, the generic drug, and the active pharmaceutical ingredient(s) of the trade name product.

Unless defined otherwise, all technical and scientific terms used herein have the same meaning as commonly understood by one of ordinary skill in the art pertinent to the methods and compositions described. The following references provide one of skill with a non-exclusive guide to a general definition of many of the terms used herein: Hale & Margham, The Harper Collins Dictionary of Biology (Harper Perennial, New York, N.Y., 1991); King & Stansfield, A Dictionary of Genetics (Oxford University Press, 4th ed. 1990); Hawley's Condensed Chemical Dictionary (John Wiley & Sons, 13th ed. 1997); and Stedmans' Medical Dictionary (Lippincott Williams & Wilkins, 27th ed. 2000). As used herein, the following terms and phrases have the meanings ascribed to them unless specified otherwise.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to +/−10%.

All references cited herein are incorporated by reference in their entirety.

The present invention is based in part upon the discovery that isolated or synthetic oligonucleotides which contain at least one analogue of cytosine incorporated in a CpG sequence can be used as therapeutics for hypo-methylating aberrantly methylated genes in human cancer disease leading to restoration of aberrantly methylated gene expression. In an embodiment, the oligonucleotides are not incorporated into the genomic DNA and are not specific to any gene sequence. In another embodiment, the oligonucleotides are chemically and enzymatically stable.

The present invention provides isolated or synthetic oligonucleotides which contain at least one analogue of cytosine incorporated in a CpG sequence of the oligonucleotide. Cytosine nucleotide analogues include but are not limited to 5-aza-cytidine, 5-aza-2′-deoxycytidine, 5-fluoro-cytidine, fluorocyclopentenylcytosine, zebularine, or deoxyzebularine. The oligonucleotides are double stranded structure, and the CpG dinucleotide is preferably located in the double stranded portion of the oligonucleotide. The oligonucleotide can be either a single stranded sequence that forms a stem-loop (hairpin) or a pair of single stranded sequences that anneal to form a double stranded sequence.

The CpG sites of the oligonucleotide can be modified and provide two general versions: un-methylated, where cytosine on the first strand is not methylated and the cytosine on the complementary strand is replaced by one of the cytosine analogues selected from but not limited to 5-aza-cytidine, 5-aza-2′-deoxycytidine, 5-fluoro-cytidine, fluorocyclopentenylcytosine, zebularine, or deoxyzebularine; and, hemi-methylated, where cytosine on the first strand is methylated (5-methyl-cytosine) and the cytosine on the complementary strand is replaced by one of the cytosine analogues selected from but not limited to 5-aza-cytidine, 5-aza-2′-deoxycytidine, 5-fluoro-cytidine, fluorocyclopentenylcytosine, zebularine, or deoxyzebularine. These oligonucleotides are not incorporated into genomic DNA and are designed specifically as trapping suicide oligonucleotides (“DNMT Trapping Oligonucleotide, “DTO”), which capture or trap DNA methyltransferases. By modifying the composition of the CpG site in the oligonucleotide it is possible to create selectivity for specific DNA methyltransferase. For instance, the hemi-methylated CpG site has higher selectivity for DNA Methyltransferase 1 (DNMT1), such as in MTC-433 described herein, over or vs. DNA Methyltransferase 3A (DNMT3A) or DNA Methyltransferase 3B (DNMT3B).

The cytosine analogs described herein include but are not limited to those shown in FIGS. 4 through 8.

FIG. 4 shows the cytosine analog, 5-fluorocytidine, which is also known by the common names, Flucytosine or 5-fluorocytidine; by the chemical name: 4-amino-5-fluoro-1,2-dihydropyrimidin-2-one, and by the trade name, Ancobon.

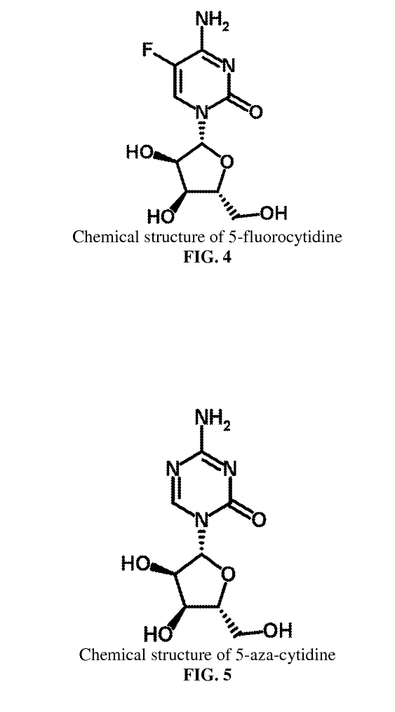

FIG. 5 shows the cytosine analog, 5-aza-cytidine, which is also known by the common names, Azacitidine or 5-aza-cytidine; by the chemical name, 4-amino-1-β-D-ribofuranosyl-1,3,5-triazin-2(1H)-one; and by the trade name: Vidaza®.

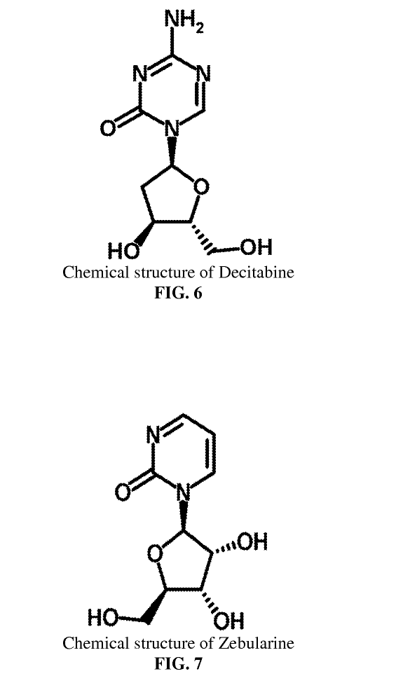

FIG. 6 shows the cytosine analog, 5-aza-2′-deoxycytidine, which is known by the common names, Decitabine; by the chemical name: 4-amino-1-(2-deoxy-b-D-erythro-pentofuranosyl)-1,3,5-triazin-2(1H)-one, and by the trade name, Dacogen®.

FIG. 7 shows the cytosine analog, Zebularine, which is also known by the common names, zebularine; and by the chemical name, 1-(β-D-Ribofuranosyl)-2(1H)-pyrimidinone.

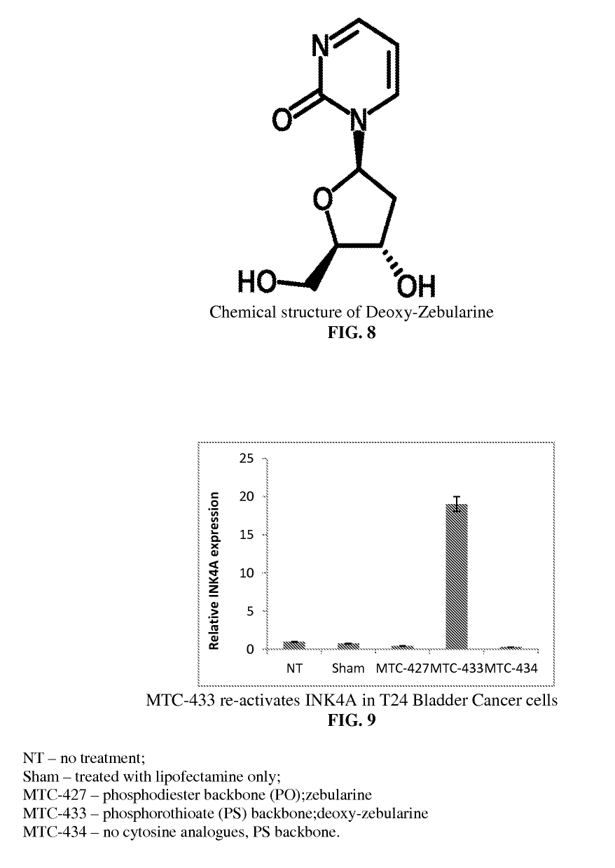

FIG. 8 shows the cytosine analog having the common name, deoxy-zebularine; and by the chemical name, 1-(β-D-deoxyribofuranosyl)-2(1H)-pyrimidinone.

FIG. 9 shows the results of MTC-433 compound re-activating a INK4A tumor suppressor gene.

In an aspect of the invention, the isolated, modified, or synthetic oligonucleotides contain at least one analogue of cytosine incorporated in a CpG sequence and which the 3′ ends are modified to increase the oligonucleotides resistance to nuclease degradation in the cell.

In another aspect of the invention, the modified oligonucleotides are provided which contain at least one analogue of cytosine incorporated in a CpG sequence and which their phosphodiester linker is replaced by artificial backbone linker to provide for the resistance to nuclease degradation in vivo. Such artificial backbone linkers can be selected from but not limited to the following: phosphorothioate linker, boranophosphate or methylphosphonate linker; the 2′-hydroxyl group of ribose can be modified to be a 2′-methoxy group, 2′-methoxyethyl group, or 2′-fluoro group. Also optionally, the sugar phosphodiester backbone can be replaced with a peptide nucleic acid (PNA) backbone where the backbone is made from repeating N-(2-aminoethyl)-glycine units linked by peptide bonds. Other types of linkers for oligonucleotides designed to be more resistant to nuclease degradation are described U.S. Pat. Nos. 6,900,540 and 6,900,301, which are herein incorporated by reference. Additional types of nucleic acids and analogs that can be incorporated into the backbone are locked nucleic acids (LNA) and morpholino nucleoside analogs. In addition, 2′ substituted nucleosides, such as 2′-o-methyl, 2′-o-methoxyethyl, 2′-fluoro, and 2′-amino substitutions can be made.

The oligonucleotides of the invention are produced by conventional means, such as for example, as shown in Example 1, herein, and pure at 70%, 75%, 80%, 85%, 90%, 95% or greater.

The invention is aimed to overcome pharmacological and toxicological issues associated with conventional hypo-methylating agents such as Dacogen® and Vidaza®. Both Vidaza® and Dacogen® are unstable chemically and enzymatically. However, the inventive oligonucleotides described herein provide the advantage that they are resistant to enzymatic degradation and are chemically more stable.

This invention is also providing for utilizing additional cytosine analogues like zebularine and deoxyzebularine, which could not be used effectively as free nucleotide hypo-methylating agents in vivo because zebularine was inefficiently phosphorylated and deoxyzebularine could not be phosphorylated at all. Incorporation of zebularine or deoxyzebularine into the oligonucleotides of the invention overcomes these difficulties enabling the use of zebularine and deoxyzebularine as a hypomethylating agents.

The isolated, modified, or synthetic oligonucleotides provided in herein also should overcome toxicities associated with the incorporation of cytosine analogues into genomic DNA as the modified oligonucleotides are designed and synthesized in such forms that they are never incorporated into genomic DNA as opposed to the free nucleoside forms of cytosine analogues which are randomly and extensively incorporated into the genomes of all dividing cells causing genome instability and genotoxicity. Isolated, modified, or synthetic oligonucleotides provide an independent target for DNA methyltransferases thus sparing the genome from potential mutagenic effects of the cytosine analogues and eliminating DNMT methyltransferase:DNA complexes that when formed on the genomic DNA lead to DNA synthesis disruption and further genomic DNA damage.

The demethylation effect of the isolated, modified, or synthetic oloigonucleotide inhibitors described herein can be measured in cell based assays known in the art and include, for example, DNMT inhibition assays (such as Examples 2-4 and 7 described herein), cell-based reporter assays (such as Example 5 described herein), animal models (such as Examples 6, 10 and 11 described herein), and re-activation of tumor suppressor genes (such as Examples 8 and 10 described herein).

Additional assays are known in the art and include measurement of 5-methylcytosine in the Mouse Erythroleukemia Cell line. See Flynn J, Fang J, Mikovits J, and Reich, N. A Potent Cell-active Allosteric Inhibitor of Murine DNA Cytosine C5 Methyltransferase. (2003) J. Biol. Chem. 278, 8238-8243. In this assay, MEL cells, are prepared as described to a density of 10⁶/ml, and treated with inhibitors along with LipofectAMINE as described by the manufacturer. The mock treatment uses TE (Tris, pH 8.0 (10 mm), and EDTA, 1 mm) in place of any inhibitor. The inhibitors or the mock are added only at the initiation of the experiment, and genomic DNA is isolated after 72 and 110 h of cell culture. 5-Methylcytosine content is determined as described. Briefly, MspI endonuclease is used to digest the genomic DNA, and the samples are then treated sequentially with calf intestinal alkaline phosphatase, T4 polynucleotide kinase (and [γ-32P]ATP), and P1 nuclease. The ³²P end-labeled cytosines, either 5-mCMP or CMP within the CpG of the MspI ends, are separated on cellulose thin layer chromatography plates. A PhophorImager (Amersham Biosciences) is used to visualize and quantify the percent 5-methylcytosine. The effects of the isolated, modified, or synthetic oligonucleotide DNMT inhibitors of the present invention will show a decrease in 5-methylcytosine, due to the hypo-methylation activities of the oligonucleotides.

Another assay measures gene methylation in human colon cancr cells: HT29 cells are plated at 50% confluence in six-well plates and treated with 5AC at 1 μm or LipofectAMINE-transfected with either test or control oligonucleotides for 48-72 h. DNA is isolated using the Qiagen blood and cell culture DNA kit (Qiagen) according to the manufacturer's instructions. Methyl-specific PCR (MSP) is performed as described. Peripheral blood lymphocytes are used as a positive control for unmethylated p16, and peripheral blood lymphocyte DNA, methylated in vitro using SssI methylase (New England Biolabs) according to the manufacturer's instructions, is used as a positive control for methylated p16. RNA is isolated using Trizol (Invitrogen). cDNA is prepared from 1 μg of RNA using a Superscript II reverse transcription system with random hexamers as primers (Invitrogen). PCR is performed using primers for p16 designed to cross a splice junction in the gene as described, on 1 μl of cDNA product. The reaction is initiated with a 3-min incubation at 94° C. followed by 35 amplification cycles (94° C. for 30 s, 58° C. for 1 min, 72° C. for 1 min) and a final 10 min extension step of 10 min following electrophoresis on a 1.5% agarose gel. The gel is stained with ethidium bromide and photographed. Expression of β-actin is used as a standard for RNA integrity and equal gel loading. See Flynn J, Fang J, Mikovits J, and Reich, N. A Potent Cell-active Allosteric Inhibitor of Murine DNA Cytosine C5 Methyltransferase. (2003) J. Biol. Chem. 278, 8238-8243. The effects of the isolated, modified, or synthetic oligonucleotide DNMT inhibitors of the present invention will show reduction, lessening, or inhibition of gene methylation, due to the hypo-methylation activities of the oligonucleotides.

Another assay that can be used to measure the effects of the isolated or synthetic oligonucleotide DNMT inhibitors of the invention uses multiplex methylation-specific PCR assay to measure methylation of ovarian cancer. See, for example, Zhanga Q, Huc G, Yangd Q, Donga R, Xiee X, Maf D, Sheng K, and Konga B. A Multiplex Methylation-Specific PCR Assay for the Detection of Early-Stage Ovarian Cancer Using Cell-Free Serum DNA. (2013) Gynecologic Oncology 130, 132-139. In this assay, the methylation status of seven candidate genes (APC, RASSF1A, CDH1, RUNX3, TFPI2, SFRP5 and OPCML) in minimal cell-free serum DNA is measured. The effects of the isolated, modified, or synthetic oligonucleotide DNMT inhibitors of the present invention will show a re-activation of gene expression, such as of one or more of these genes, due to the hypo-methylation activities of the oligonucleotides.

The DNMT inhibiting oligonucleotides described herein can be produced by a variety of methods known to one skilled in the art, and is also described herein in Example 1. One method uses phosphoramidite solid-phase synthesis method using phosphoramidite building blocks derived from protected 2′-deoxynucleosides (dA, dC, dG, and T), ribonucleosides (A, C, G, and U), or chemically modified nucleosides. Essentially, the building blocks are sequentially coupled to the growing oligonucleotide chain in the order of the oligonucleotide sequence. The process can be fully automated. When the chain assembly is completed, the product is released from the solid phase to solution, de-protected, and collected. Oligonucleotides of the present invention include oligos with a phosphodiester (or phosphorothioate) links (or internucleoside links) incorporating 5-aza-cytidine, zebularine, or 5-methyl-cytidine, etc., subunits. For example the building blocks for 5-aza-cytidine 5′-Dimethoxytrityl-N4-dimethylformamidine-5,6-dihydro-5-aza-2′-deoxyCytidine, 3′-[(2-cyanoethyl)-(N,N-diisopropyl)]-phosphoramidite.

The oligonucleotides of the invention can be administered by any route, preferably in the form of a pharmaceutical composition adapted to such a route. The compounds and compositions can be, for example, administered orally, parenterally, intraperitoneally, intravenously, intraarterially, transdermally, sublingually, intramuscularly, rectally, topically, transbuccally, intranasally, liposomally, via inhalation, vaginally, intraoccularly, via local delivery (for example by a catheter or stent), subcutaneously, intraadiposally, intraarticularly, infusion, or intrathecally.

Generally, the dosage of administered oligonucletoides compositions, will vary depending upon such factors as the subject's age, weight, height, sex, general medical condition and previous medical history. For example, the oligonucleotides can be administered at a range of about 10 to about 100 mg/m2 intravenously by infusion for 1 to 10 hours, every 4 to 10 hours, for 1 to 10 days and repeated every 3 to 6 weeks. A lower or higher dosage also may be administered as circumstances dictate. As an example, and for illustration only, Dacogen® has been administered in humans at ˜−15 mg/m2 IV infusion for 3 hours, every 8 hours for 3 days—repeat every 6 weeks. Similarly, the drug formulation and schedule of administration can be diluted in 500 mL of normal saline for intravenous injection and infused as a 3-hour infusion on 3 consecutive days as a loading dose, followed by weekly maintenance doses, with doses of about 100 mg to about 1000 mg, for example, of about 750 mg.

See, for example, Talbot D C, Ranson M, Davies J, Lahn M, Callies S, André V, Kadam S, Burgess M, Slapak C, Olsen A L, McHugh P J, de Bono J S, Matthews J, Saleem A, Price P., Tumor Survivin is Downregulated by the Antisense Oligonucleotide LY2181308: a Proof-of-Concept, First-in-Human Dose Study., Tumor Survivin is Downregulated by the Antisense Oligonucleotide LY2181308: a Proof-of-Concept, First-in-Human Dose Study. Clin Cancer Res. 2010 Dec. 15; 16(24):6150-8. Epub 2010 Nov. 1. The oligonucleotides can be diluted in aqueous solution, saline solution, or other solutions.

The amount of isolated, modified, or synthetic oligonucleotide DNMT inhibitor in tissue can be measured by capillary electrophoresis. For example, tissues are minced while still frozen, and an aliquot weighed into a microcentrifuge tube. A phosphorothioate oligonucleotide is added as the internal standard immediately after each sample is aliquoted. Following the addition of 0.5 ml of digestion buffer consisting of 0.5% Nonidet P-40 with 20 mM Tris-HCl, pH 8.0, 20 mM EDTA, and 100 mM NaCl, the tissues are homogenized in a Savant Bio 101 tissue disruptor. An aliquot of proteinase K is then added so that the final concentration is 2.0 mg/ml, and the samples incubated overnight at 37° C. The samples are then extracted with phenol-chloroform-isoamylalcohol (25:24:1) to remove proteins and lipids; nucleic acids remain in the aqueous phase. The phenol-chloroform isoamyl alcohol layer is back-extracted with 500 ml of distilled H₂O, and the aqueous phases pooled. Samples are then evaporated to dryness, resuspended in 200 μl of concentrated ammonium hydroxide, and incubated at 55° C. overnight. They are then re-evaporated to dryness and resuspended. Capillary gel electrophoresis separations for tissue samples is performed with an electrophoresis device (e.g. Beckman P/ACE). The gel-filled capillary column contains 12% polyacrylamide with 8.3 M urea, and the running buffer (100 mM Tris-Borate, pH 8.5). Separation is achieved by running the gel at 50° C. and 550 V per cm. Oligomers eluting from the column are detected by ultraviolet absorption at a wavelength of 260 nm. The oligonucleotides are quantitated by determining the corrected area (area under the UV absorbance curve divided by migration time) and dividing by the corrected area of the internal standard peak. See, Geary R, Leeds J, Fitchett J, Burckin T, Truong L, Spainhour C, Creek M, and Levin A., Pharmacokinetics and Metabolism in Mice of a Phosphorothioate Oligonucleotide Antisense Inhibitor of C-raf-1 Kinase Expression Drug Metabolism and Disposition 25: 1272-1281 (1997).

The oligonucleotides described herein can be used to minimize, limit, reduce or inhibit aberrant methylation in diseases such as acute-myeloid leukemia (AML), chronic myeloid leukemia (CML), myelodysplastic syndromes (MDS), cancers of the liver and kidney and hyperproliferative diseases and syndromes of the liver and kidney, as well as lung and ovarian cancer and diseases and syndromes of ovarian cell proliferation, as well as cancers and cell-proliferative disorders or diseases of breast, colorectal or pancreatic cancer.

Acute myeloid leukemia (AML) is the most common type of acute leukemia that occurs in adults. There are several inherited genetic disorders and immunodeficiency states that are associated with an increased risk of AML, including disorders with defects in DNA stability, leading to random chromosomal breakage, such as, Fanconi's anemia, Li-Fraumeni kindreds, ataxia-telangiectasia, and X-linked agammaglobulinemia, and Bloom's syndrome.

Acute promyelocytic leukemia (APML) is one of the subgroups of AML, and is characterized by promyelocytic blasts containing the 15; 17 chromosomal translocation. This translocation leads to the generation of the fusion transcript comprised of the retinoic acid receptor and a sequence PML.

Chronic myelogenous leukemia (CML) is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML.

Acute lymphoblastic leukemia (ALL) is a heterogenerous disease with distinct clinical features displayed by various subtypes. Reoccurring cytogenetic abnormalities have been demonstrated in ALL. The most common cytogenetic abnormality is the 9; 22 translocation. The resultant Philadelphia chromosome represents poor prognosis of the patient.

The myelodysplastic syndromes (MDS) are heterogeneous clonal hematopoietic stem cell disorders grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS typically develop complications related to anemia, neutropenia (infections), or thrombocytopenia (bleeding). Generally, from about 10% to about 70% of patients with MDS develop acute leukemia.

The effect of administering the hypo-methylating oligonucleotides can be measured in vivo by a reduction, inhibition, or minimization, or methylation in genomic DNA. Assays to measure DNA methylation are known in the art and are described by the Examples herein. Physiological symptoms may not be present, but if they are would include a reduction, minimization, limitation, or inhibition of cancer-associated symptoms, such as weight loss, uncontrolled cell growth, fevers, chills, night sweats, fatigue, nausea, pain, and other flu-like symptoms, or reduction in size of the liver, kidney or spleen.

A pharmaceutical composition comprising or consisting of the oligonucleotides can be furnished in liquid form, in an aerosol, or in solid form. Liquid forms, are illustrated by injectable solutions and oral suspensions. Exemplary solid forms include capsules, tablets, and controlled-release forms. The latter form is illustrated by miniosmotic pumps and implants (Bremer et al., Pharm. Biotechnol. 10:239 (1997); Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)).

As another example, liposomes provide a means to deliver the oligonucleotides to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see, generally, Bakker-Woudenberg et al., Eur. J. Clin. Microbiol. Infect. Dis. 12 (Suppl. 1):S61 (1993), Kim, Drugs 46:618 (1993), and Ranade, “Site-Specific Drug Delivery Using Liposomes as Carriers,” in Drug Delivery Systems, Ranade and Hollinger (Eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 micrometers to greater than 10 micrometers. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576 (1989)). Moreover, it is possible to control the therapeutic availability of the encapsulated agent by varying liposome size, the number of bilayers, lipid composition, as well as the charge and surface characteristics of the liposomes. Methods of delivering nucleic drugs also include formulation of the DNMT inhibiting oligonucleotides in a formulation of lipid-particles and cyclodextrin-adamantane polyethylene glycol particles. See, for example, Kole, R., et al., RNA therapeutics: beyond RNA interference and antisense oligonucleotides, Nat. Rev. Drug Discovery. Vol. 11: 125-140 (2012).

Various references, including patent applications, patents, and scientific publications, are cited herein, the disclosures of each of which is incorporated herein by reference in its entirety.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The invention is further illustrated by the following non-limiting examples.

### EXAMPLES

### Example 1

### Preparation of Oligonucleotide-Based Compounds

The oligonucleotide-based compounds of the invention were chemically synthesized using phosphoramidite chemistry on an automated DNA/RNA synthesizer by Bio-Synthesis Inc. (Lewisville, Tex.) or by Generi Biotech (s.r.o., Machkova 587, Hradec Kralove, CZ-50011, Czech Republic). Modified DNA bases were incorporated at defined positions into oligonucleotides during automated synthesis. The modified base, 5,6-dihydro-5-aza-cytidine, was incorporated using the 5-aza-5,6-dihydro-dC-CE Phosphoramidite (Glen Research, Sterling Va.). The modified base, 2-pyrimidone ribonucleoside (zebularine—a cytidine analog lacking an amino group), was incorporated using Zebularine-CE Phosphoramidite (Glen Research, Sterling Va.). Oligonucleotides were purified, quantitated by UV spectrophotometry, quality checked by MALDI-TOF mass spectrometry, lyophilized and shipped. Oligonucleotides were reconstituted in an aqueous solution and used in studies. The sequences of the oligonucleotides are shown below in Table 1.

TABLE 1
Sequences of Oligonucleotides with cytosine analogues

| | | | SEQ |
|---|---|---|---|
| | | | ID |
| Oligo | Sequence | Modification | NO: |
| MTC- | 5′CCTATGCGATCGAGTTTTCT[x]GAT[x]GCATAGG3′ | x = 5-aza-5,6- | 3 |
| 422 | | dihydro-dC | |
| MTC- | 5′CCTATGCGATCGAGTTTTCT[z]GAT[z]GCATAGG3′ | z = zebularine | 4 |
| 423 | | (pyrimidin-2- | |
| | | one | |
| | | ribonucleoside) | |
| MTC- | 5′CCTATG[M]GAT[M]GAGTTTTCT[x]GAT[x]GCATAGG3′ | M = 5- | 5 |
| 422m | | methylcytosine; | |
| (also | | x = 5-aza-5,6- | |
| called | | dihydro-dC | |
| MTC- | | | |
| 425) | | | |
| MTC- | 5′CCTATG[M]GAT[M]GAGTTTTCT[z]GAT[z]GCATAGG3′ | M = 5- | 6 |
| 423m | | methylcytosine; | |
| (also | | z = zebularine | |
| called | | (pyrimidin-2- | |
| MTC- | | one | |
| 427) | | ribonucleoside) | |
| MTC- | 5′GTACATG[X]GCTCCAGA3′ | X = 5-aza-5,6- | 7 |
| 424F | | dihydro-dC | |
| MTC- | 5′TCTGGAGCGCATGTAC3′ | | 8 |
| 424R | | | |
| MTC- | 5′GTACATG[x]GCTCCAGA3′ | x = 5-aza-5,6- | 9 |
| 429F | | dihydro-dC | |
| MTC- | 5′TCTGGAG[M]GCATGTAC3′ | M = 5- | 10 |
| 429R | | methylcytosine | |
| MTC- | 5′CCTATGCGATCGAGTTTTCT[dz]GAT[dz]GCATAGG3′ | [dz] = deoxy- | 11 |
| 432 | | zebularine | |
| MTC- | 5′CCTATG[M]GAT[M]GAGTTTTCT[dz]GAT[dz]GCATAGG3′ | [M] = 5- | 12 |
| 433 | | methylcytosine; | |
| | | [dz] = deoxy- | |
| | | zebularine | |
| MTC- | 5′CCTATGGCATGCAGTTTTCTGCATGCCATAGG3′ | | 13 |
| 434 | | | |
| MTC- | 5′CCTATG[M]GAT[M]GAGTTTTCT[dz]GAT[dz]GCATAGG3′ | M = 5- | 14 |
| 443 | | methylcytosine; | |
| | | [dz] = deoxy- | |
| | | zebularine | |

### Example 2

### DNA (Cytosine-5) Methyltransferase 1 Assay (DNMT1)

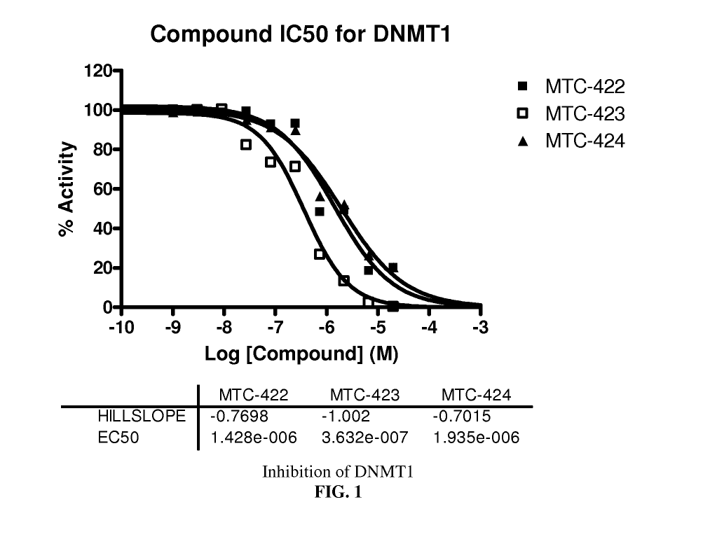

DNA Methyltransferase 1 (DNMT1) was essentially assayed as described in Tollefsbol, T. O. and Hutchison III, C. A.: J. Biol. Chem. (1995), 270:18543-50 with the following modifications. Assay reaction was 5 μl volume, the DNA substrate was poly (dI:dC) 0.001 mg/ml and co-factor S-AdoMet at 1.0 μM; human DNMT1 was added at 25 nM. Reaction: S-adenosyl-L-[methyl-3H] methionine+DNA=S-adenosyl-L-homocysteine+DNA 5-[methyl-3H]-cytosine. S-AdoHCy was used as a positive control for the inhibition reaction. Oligonucleotide compounds were tested in 10-dose IC50 mode with 3-fold serial dilution starting at 20 μM. DNMT1 inhibition results shown in Table 2 and FIG. 1 are displayed as % activity negative DMSO control reaction.

TABLE 2
Inhibition of DNMT1 with Oligonucleotides

| | | | MTC-424 |
|---|---|---|---|
| | | | (SEQ ID NO: 5 and |
| | MTC-422 | MTC-423 | SEQ ID NO: 6) - |
| Conc (M) | (SEQ ID NO: 3) | (SEQ ID NO: 4) | control oligo w/5-aza |
| 2.00E−05 | 20.15 | 0.32 | 20.30 |
| 6.67E−06 | 18.43 | 2.34 | 26.33 |
| 2.22E−06 | 49.11 | 13.21 | 52.14 |
| 7.41E−07 | 48.29 | 26.78 | 56.27 |
| 2.47E−07 | 93.00 | 71.16 | 89.83 |
| 8.23E−08 | 92.85 | 73.49 | 91.12 |
| 2.74E−08 | 99.33 | 82.21 | 94.91 |
| 9.14E−09 | 98.88 | 100.39 | 98.09 |
| 3.05E−09 | 100.20 | 99.97 | 99.21 |
| 1.02E−09 | 100.11 | 99.74 | 98.81 |
| DMSO | 100.35 | 100.02 | 99.63 |
| Ctrl | | | |
| | −0.77 | −1.00 | −0.70 |
| IC₅₀ | 1.43E−06 | 3.63E−07 | 1.94E−06 |

Results as illustrated in FIG. 1. Hairpin oligo's containing a 5-aza-5,6-dihydro-dC cytosine analog show 50% inhibition of DNMT1 activity at an estimated concentration of 1.4 uM, the zebularine containing hairpin oligonucleotide at an estimated concentration of 363 nM and the double stranded oligonucleotide containing the 5-aza-5,6-dihydro-dC at an estimated concentration of 1.94 uM. Thus, the oligonucleotides of the invention can be used to reduce, limit, minimize DNMT1 activity, which would result in an improvement of aberrant methylation of genes, or restoration of expression of aberrantly silenced genes, such as tumor suppressor genes and the like.

### Example 3

### DNA (Cytosine-5) Methyltransferase 3A Assay (DNMT3A)

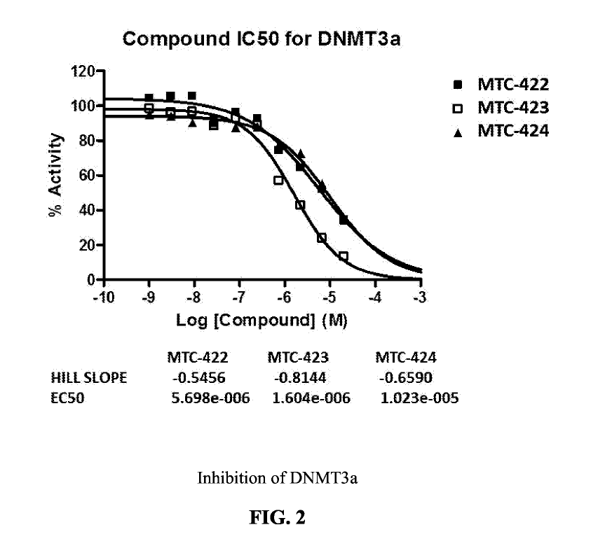

DNA Methyltransferase 3A (DNMT3A) was essentially assayed as described in Tollefsbol, T. O., et al. as described above with the following modifications. Assay reaction was 5 μl volume, the DNA substrate was Lambda DNA at 0.0075 mg/ml and co-factor S-AdoMet at 1.0 μM; human DNMT3A was added at 25 nM. Reaction: S-adenosyl-L-[methyl-3H] methionine+DNA=S-adenosyl-L-homocysteine+DNA 5-[methyl-3H]-cytosine. We used S-AdoHCy as a positive control for the inhibition reaction. Oligonucleotide compounds were tested in 10-dose IC50 mode with 3-fold serial dilution starting at 20 μM. DNMT1 inhibition results shown in Table 3 and FIG. 2 are displayed as % activity negative DMSO control reaction.

TABLE 3
Inhibition of DNMT3A with Oligonucleotides

| | | | MTC-424 |
|---|---|---|---|
| | | | (SEQ ID NO: 5 and |
| | MTC-422 | MTC-423 | SEQ ID NO: 6) - |
| Conc (M) | (SEQ ID NO: 3) | (SEQ ID NO: 4) | control oligo w/5-aza |
| 2.00E−05 | 34.69 | 13.49 | 34.41 |
| 6.67E−06 | 51.88 | 23.99 | 55.06 |
| 2.22E−06 | 64.40 | 42.81 | 72.65 |
| 7.41E−07 | 74.35 | 57.01 | 77.29 |
| 2.47E−07 | 92.69 | 88.96 | 87.71 |
| 8.23E−08 | 96.56 | 92.83 | 87.33 |
| 2.74E−08 | 92.21 | 88.42 | 90.57 |
| 9.14E−09 | 105.44 | 96.68 | 90.12 |
| 3.05E−09 | 105.70 | 96.38 | 93.98 |
| 1.02E−09 | 104.33 | 98.30 | 94.96 |
| DMSO | 100.23 | 99.88 | 99.89 |
| Ctrl | | | |
| | −0.55 | −0.81 | −0.66 |
| IC₅₀ | 5.70E−06 | 1.60E−06 | 1.02E−05 |

Results show that as illustrated in FIG. 2 the hairpin oligonucleotide containing a 5-aza-5,6-dihydro-dC cytosine analoge show 50% inhibition of DNMT1 activity at an estimated concentration of 5.7 uM, the zebularine containing hairpin oligonucleotide at an estimated concentration of 1.6 uM and the double stranded oligonucleotide containing the 5-aza-5,6-dihydro-dC at an estimated concentration of 10.2 uM. Thus, the oligonucleotides of the invention can be used to reduce, limit, minimize DNMT3A activity, which would result in an improvement of aberrant methylation of genes, or restoration of expression of aberrantly silenced genes, such as tumor suppressor genes and the like.

### Example 4

### DNA (Cytosine-5) Methyltransferase 3B Assay (DNMT3B)

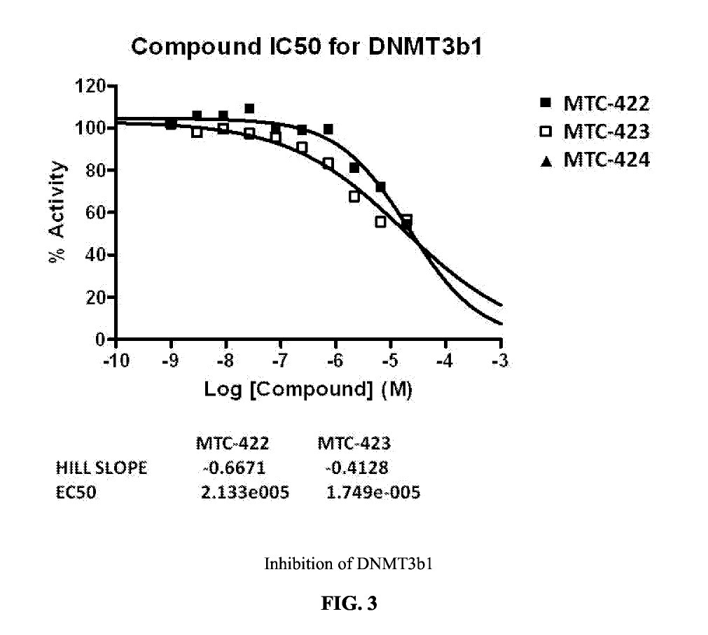

DNA Methyltransferase 3B (DNMT3B) was essentially assayed as described in Tollefsbol, T. O., et al. as described above with the following modifications. Assay reaction was 5 μl volume, the DNA substrate was Lambda DNA at 0.0075 mg/ml and co-factor S-AdoMet at 1.0 μM; human DNMT3B was added at 25 nM. Reaction: S-adenosyl-L-[methyl-3H] methionine+DNA=S-adenosyl-L-homocysteine+DNA 5-[methyl-3H]-cytosine. We used S-AdoHCy as a positive control for the inhibition reaction. Oligonucleotide compounds were tested in 10-dose 1050 mode with 3-fold serial dilution starting at 20 μM. DNMT1 inhibition results shown in Table 4 and FIG. 3 are displayed as % activity negative DMSO control reaction.

TABLE 4
Inhibition of DNMT3B with Oligonucleotides

| | | | MTC-424 |
|---|---|---|---|
| | | | (SEQ ID NO: 5 and |
| | MTC-422 | MTC-423 | SEQ ID NO: 6) - |
| Conc (M) | (SEQ ID NO: 3) | (SEQ ID NO: 4) | control oligo w/5-aza |
| 2.00E−05 | 54.20 | 56.57 | 68.21 |
| 6.67E−06 | 71.91 | 55.41 | 82.42 |
| 2.22E−06 | 81.20 | 67.48 | 87.92 |
| 7.41E−07 | 99.23 | 83.12 | 99.17 |
| 2.47E−07 | 98.85 | 90.67 | 101.96 |
| 8.23E−08 | 99.42 | 95.30 | 101.57 |
| 2.74E−08 | 109.12 | 97.22 | 104.85 |
| 9.14E−09 | 105.42 | 99.60 | 99.15 |
| 3.05E−09 | 105.69 | 97.79 | 103.20 |
| 1.02E−09 | 101.33 | 102.45 | 95.65 |
| DMSO | 101.07 | 100.84 | 98.09 |
| Ctrl | | | |
| | −0.67 | −0.41 | |
| IC₅₀ | 2.13E−05 | 1.75E−05 | |

Results show that as illustrated in FIG. 3 the hairpin oligonucleotide containing a 5-aza-5,6-dihydro-dC cytosine analoge show 50% inhibition of DNMT1 activity at an estimated concentration of 21.3 uM, the zebularine containing hairpin oligonucleotide at an estimated concentration of 17.5 uM and the double stranded oligonucleotide containing the 5-aza-5,6-dihydro-dC did not inhibit the activity sufficiently to estimate the 50% inhibition concentration. Thus, the oligonucleotides of the invention can be used to reduce, limit, minimize DNMT3B activity, which would result in an improvement of aberrant methylation of genes, or restoration of expression of aberrantly silenced genes, such as tumor suppressor genes and the like.

### Example 5

### Treatment of Cells with the DNMT Inhibitor Modified Oligonucleotide Compounds

Cells are seeded at desired density in 96 well plates (50-70% confluency). Allow cells to recover at 37 degrees C., 5% Co2 for 24 hrs.

Prepare Oligonucleotide Complexes:

Liposome/DNA complexes must be prepared in serum-free media. Cations in the serum will form complexes with the transfection reagent, competing with the DNA Oligonucleotides are purified by ethanol precipitation, or washing on a spin column and diluted in serum free, antibiotic free media to a final concentration of 1 ug/100 ul of media. Aliquots of transfection reagent (volumes of 1, 2, 4, and 6 ul) are prepared in 100 ul total vol of serum free, antibiotic free media. The oligonucleotide solution and transfection reagent solutions are combined and mixed gently by tapping with fingertip. The resulting solution is incubated for 15-45 minutes at room temperature to allow complex formation.

To optimize transfection efficiency, the ratio of transfection reagent to oligonucleotide concentration is titrated. Subsequently the dose of transfection reagent/oligonucleotide can be optimized in a dose response experiment.

Treatment:

Aspirate media from cells and replace with transfection reagent. Excessive exposure to the transfection reagent can cause toxicity or cell death. Recommended transfection start times are 6-8 hours, serum-free, but the ideal time may be greater than that. For adherent cells in serum containing media, treat cells overnight (app 16 hours) as a starting point in a standard protocol. Repeat treatment as determined empirically.

Harvest cells/DNA-cells can be lysed directly while still in the plate using g-DNA miniprep kits, or trypsinized allowing counting of the cells prior to further use.

Reporter Assays:

To determine the effects of treatment, cellular DNA is isolated and the level of DNA methylation is measured using various techniques. For the measurement of methylation at specific loci, including but not limited to, INK4A (p16), Septin9 SEPT9: RASSF1A, APC, CDKN2B, BRCA1, MGMT, DAPK, TMS1, CDC2, SFRP1, TIMP-3, CACNA1G, IGF2, NEUROG1, RUNX3, SOCS1, BRAF, KRAS, RARB2, MLH1, the DNA sample is treated with bisulfite to convert unmethylated cytosines to uracils, and the DNA sample is then subject to methylation specific PCR assays. For the measurement of genome wide effects on cytosine methylation, techniques such as quantitative 5-methylcytosine ELISA or quantitative methylation specific LINE1 real time PCR assays are applied.

### Example 6

### Animal Model for DNA Inhibitor Analysis

Preparation of Inhibitors:

Doses of appropriate concentrations are prepared for 0.25 mL injections. For compounds soluble in aqueous solution, such as oligonucleotides, the compounds are dissolved in Phosphate Buffered Saline (PBS). To improve stability in the mouse model, oligonucleotides are also prepared incationic liposomes and injected. Oligonucleotide doses range from 100 ng/dose to 1 mg/dose.

Studies include 5-Aza-2 deoxycytidine as a positive control, prepared at a dose of 5 mg/kg in PBS, and either PBS alone as a negative control.

Mouse Model:

The EJ6 human bladder cancer cell or similar cancer cell lines can be used for animal model studies. EJ6 cells (5×105/injection) suspended in PBS are inoculated subcutaneously into the right and left back (along the midaxillary lines) of 4- to 6-week-old female BALB/c athymic nude-Foxn1nu mice available for example from Harlan Laboratories, San Diego, Calif. After 2-3 weeks and after macroscopic tumors (50-200 mm3) form, treatment injections are initiated. Tumors are measured with calipers, and tumor volumes calculated. Mice are weighed at the beginning and end of treatment to determine toxicity. The percent weight change for each mouse was calculated.

Treatments are by intraperitoneal (IP) or intra-venous (IV) injection with doses administered daily or on a multiday schedule over a period of 5-28 days depending on the experiment. Animals are sacrificed 24 hrs following the last treatment.

At this time, tumors are removed and each tumor divided into two separate portions. One portion is immediately homogenized in TRIzol reagent (Invitrogen, Carlsbad, Calif.) for RNA extraction, and the other portion immediately frozen in liquid nitrogen for DNA extraction later. Genomic DNA and RNA are used for analysis of the methylation status of the INK4A (p16) promoter as well as a panel of other promoter regions (including but not limited to: RASSF1A, APC, CDKN2B, BRCA1, MGMT, DAPK, TMS1, CDC2, SFRP1, TIMP-3, CACNA1G, IGF2, NEUROG1, RUNX3, SOCS1, BRAF, KRAS, RARB2, MLH1) by real time methylation specific PCR, sequencing or other standard methods, and gene expression is assessed by real time RT-PCR, respectively. Additionally, total methylation status is assessed by analysis of the repetitive DNA element LINE1, and by enzyme linked immunsorbent assay and quantitative mass spectrometry analysis of 5-methylcytosine. In some experiments additional mouse tissues are collected and prepared as above to study the de-methylating effects of the treatment. Methods to analyze or measure DNA methylation are known in the art. See, for example, Rocha, M. S. et al., Clin Chem Lab Med 2010; 48(12):1793-1798.

### Example 7

### Comparison of Oligonucleotide Inhibitors of DNMT in DNMT Inhibition Assay

Using procedures similar to the DNMT inhibition assays described in Examples 2, 3 and 4, above, illustrative compounds MTC-422, MTC-425, MTC-423, MTC-427, MTC-424, MTC-429, MTC-432, MTC-433, and MTC-434 were tested. The DNMT inhibition assay was based on measuring incorporation of tritiated methyl group from S-Adenosyl methionine to substrate DNA; IC50 value was calculated based on testing 10-dose 3-fold serial dilution of the inhibitor starting at 100 uM. The results are shown in Table 5, below:

TABLE 5
Comparison of Oligonucleotide Inhibitors in DNMT Inhibition Assay

| | | | DNMT1 | DNMT3A | DNMT3B |
|---|---|---|---|---|---|
| Compound | Structure | Features | IC50 | IC50 | IC50 |
| MTC-422 | hairpin | Unmethylated/5-aza- | 5.52 uM | 5.70 uM | 21.3 uM |
| | | cytidine; | | | |
| | | Phosphodiester backbone | | | |
| MTC-425 | hairpin | Methylated/5-aza- | 504 nM | 6.69 uM | >100 uM |
| | | cytidine; | | | |
| | | Phosphodiester backbone | | | |
| MTC-423 | hairpin | Unmethylated/Zebularine; | 348 nM | 1.5 uM | 18.9 uM |
| | | Phosphodiester backbone | | | |
| MTC-427 | hairpin | Methylated/Zebularine; | 295 nM | 1.52 uM | 6.20 uM |
| | | Phosphodiester backbone | | | |
| MTC-424 | Double | Unmethylated/5-aza- | 1.43 uM | 10.2 uM | >100 uM |
| | stranded | cytidine; | | | |
| | | Phosphodiester backbone | | | |
| MTC-429 | Double | Methylated/5-aza- | 906 nM | >50 uM | inactive |
| | stranded | cytidine; | | | |
| | | Phosphodiester backbone | | | |
| MTC-433 | Hairpin | Methylated/deoxy- | 4.22 nM | — | — |
| | | Zebularine;; | | | |
| | | Phosphorothioate | | | |
| | | backbone | | | |
| MTC-432 | Hairpin | Unmethylated/deoxy- | 8.3 nM | — | — |
| | | Zebularine;; | | | |
| | | Phosphorothioate | | | |
| | | backbone | | | |
| MTC-434 | Hairpin | No CpG sites/No | NA | — | — |
| | | cytosine analogues/ | | | |
| | | Phosphorothioate | | | |
| | | backbone | | | |

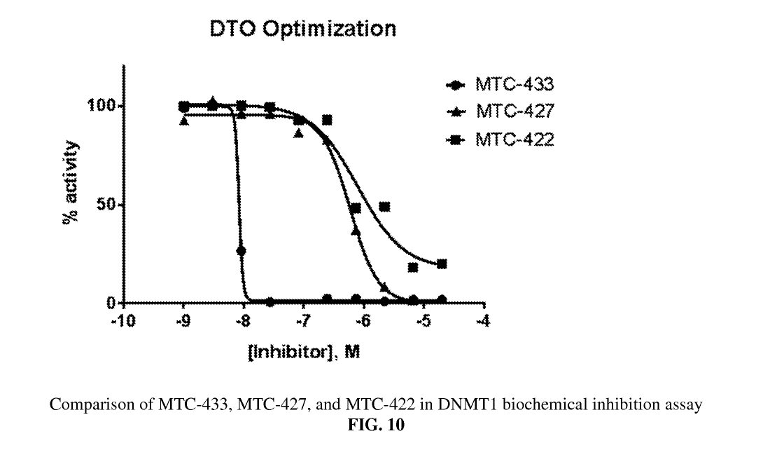

Results show that MTC-427 selectively inhibited DNMT1 over DNMT3A and DNMT3B and that MTC-433 inhibits DNMT1 better at 4.22 nM than MTC-427 at 295 nM. Results also show that MTC-433 inhibits DNMT1 better at 4.2 nM than MTC-432 at 8.3 nM. Data from the comparison of MTC-433, MTC-427, and MTC-422 in DNMT1 biochemical inhibition assay are also shown in FIG. 10.

### Example 8

### Re-Activation of Tumor Suppressor Gene INK4A in T24 Bladder Cancer Cells

Summary:

INK4A is a prototypical tumor suppressor gene used as a gold standard in DNA methylation studies. Oligonucleotide inhibitors of DNMT (MTC compounds) were delivered to T24 bladder cancer cells using lipofectamine. RNA was extracted from the cells that had been treated with MTC compounds for 6 hours on 3 consecutive days. INK4A transcript levels were determined by quantitative real-time PCR and were measured as relative expression levels normalized to 18S rRNA expression.

Transfection Protocol:

Day 1: cells are seeded at 5×10⁴/well in a 48 well plate (0.7 cm² surface area) and allowed to grow for 16 hours at 37° C., 5% CO₂. On Day 2 cells are treated with serum free/antibiotic free media containing transfection reagent with/without MTC compounds. Transfection complexes are generated by diluting Lipofectamine 1 ul into 12.5 ul serum free/antibiotic free media; MTC compounds are also diluted 1 ul into 12.5 ul serum free/antibiotic free media, the MTC compound stock being at a concentration of 100 uM (final concentration of 0.1 uM). The 12.5 ul aliquots are combined and allowed to incubate at room temperature for 5 minutes, then diluted into 1 ml of serum free/antibiotic free media. The media in the wells of the 48 well plate are replaced with the transfection reagent containing serum free/antibiotic free media (200 ul/well), and after 6 hours incubation at 37° C., 5% CO₂, the transfection media is replaced with complete media containing 10% FBS and antibiotic. The cells are incubated at 37° C., 5% CO₂ overnight and the process is repeated (Day 3, Day 4) for a total of 3 repetitions. The cells are then allowed to recover for 3 days and harvested.

Re-Expression Assay:

RNA is harvested using Trizol (Invitrogen, San Diego, Calif.) according to the manufacturer's protocol. cDNA synthesis is done with the High Capacity cDNA kit (Invitrogen, San Diego, Calif.) again according to manufacturer's recommendations. The expression of genes of interest is evaluated using taqman based RT-PCR gene specific kits (Invitrogen, San Diego, Calif.) with ribosomal RNA (18s) used as a loading control.

In this assay, MTC-427, MTC-433, and MTC-434 were compared against a no treatment group (NT), a lipofectamine-only treated group (Sham). The results are shown in FIG. 9.

Conclusions:

MTC-433 re-activates INK4A expression silenced by aberrant methylation almost 20 fold better than MTC-427 and MTC-434, indicating a stable compound resistant to nuclease degradation; neither MTC-427 (PO backbone sensitive to nucleases) nor MTC-434 (DTO control) were able to re-activate INK4A.

In this assay, a time course treating T24 cells with MTC-432 and MTC-433 over a 3 day period showed 2 fold re-activation of INK4A after only 1 day of treatment, more than 2 fold induction after 2 days of treatment, and more than 20 fold re-activation after 3 days of treatment.

### Example 9

### Cytotoxicity Assay in T24 Bladder Cancer Cells

Summary: T24 cells were treated with MTC compounds for 6 hours (1 d) or for 6 hours on 3 consecutive days (3 d) and cell toxicity was assessed with MTT assay.

Cells are seeded in 96 well plates at a concentration of 1×10⁴ cells/well and allowed to grow for 16 hours at 37° C., 5% CO₂. Cells are treated in exactly the same manner as described in the tansfection protocol for three days, then subjected to the MTT assay. Briefly, 10 ul of MTT reagent is added to each well already containing 100 ul of media. After 2 hours at 37° C., 5% CO₂ 200 ul of detergent reagent is added and the cells are again incubated at 37° C., 5% CO₂ for 2 hours. Absorbance is then read at 570 nm. A higher absorbance indicates more viable cells.

Controls (5-Azacytidine, Zebularine, Etc.)

Cells are set up as in the transfection protocol, but drugs are not delivered in transfection complexes. MTC compounds are diluted in stock concentrations in DMSO, and these are diluted to the desired concentrations in complete media containing FBS and antibiotics. Cells are treated with MTC compounds containing media for 3 days, then allowed to recover along with the cells that have been subjected to transfection. Harvesting of RNA, etc, is done in the same manner as with those cells that had been transfected.

In this assay, MTC-432, and MTC-433, were compared against a no treatment group (NT), a lipofectamine-only treated group (Sham).

Conclusions:

MTC oligonucleotide compounds showed no difference in cell toxicity when compared to delivery vehicle (lipofectamine).

### Example 10

### Re-Activation of Tumor Suppressor Genes Rassf1 and APC in HEP2 Liver Cancer Cells

Summary:

Using a protocol similar to Example 8 above, HepG2 cells were transfected with MTC-433 and MTC-434 and Rassf1 and APC gene re-activation was measured with RT-PCR after a 3 day treatment. In this assay, MTC-433 was compared to a lipofectamine only (Sham) group, MTC-434 (no CpG site, no cytosine analogues) and Zebularine.

Conclusions:

1) MTC-433 showed significantly higher levels of reactivation of both Rassf1 and APC.

2) MTC-433 showed equivalent induction of Rassf1 expression after 3 days of treatment as Zebularine (free nucleoside). MTC-432 showed no difference in Rassf1 expression after 3 days when compared to the lipofectamine control.

3) MTC-433 showed substantial induction of APC expression after 3 days of treatment, whereas MTC-432 and Zebularine (free nucleoside) reactivate APC only moderately.

### Example 11

### A Xenograph Efficacy Study of MTC433 in a Mouse Orthotopic Liver Model

In this model, drug efficacy is measured through tumor and body weight, survival, a-fetal protein (AFP) serum levels, and biomarkers. Hep2G cells are directly injected into the liver of a mouse (Nude or Scid, female, 4-6 weeks) and the MTC compound is dosed starting at about day 14 post-implantation. The mice are treated for 2 weeks once daily and sacrificed 48 hours after the last dose.

Treatment groups are: vehicle, MTC-433, sorafenib, and Combination (MTC-433 and sorafenib). Dosages of MTC-433 are 5 mg/kg and dosing is given i.v. via tail vein injection daily for MTC-433 and by oral gavage once daily at 30 mg/kg for sorafenib. Necropsies are performed at Day 28.

Measurements of gross pathology at necropsy, organ weights and RNA procedures from liver, spleen kidney, and ovaries are taken. In addition tissue analysis includes liver weight and tumor volume, tumor histology, and biomarker analysis.

### Example 12

### A Xenograph Efficacy Study of MTC433 in Mouse Subcutaneous Liver Model

In this model, drug efficacy is measured through tumor size and biomarkers in female mice (Nude or Scid, 4-6 weeks).

HepG2 cells in matrigel are subcutaneously implanted. Drug dosing is started about 4 days after implantation and mice are treated for about 3 weeks, once daily. Tumor size is measured with calipers every 3 days. Mice are sacrificed at 48 hr after last dose.

Treatment groups are vehicle and MTC-433, which is dosed at 5 mg/kg by i.v. via tail vein (200 ul total volume/injection) once daily for 28 days.

Study observations include body weights and temperatures, tumor measurement, blood analyses, and tissues at necropsy (gross pathology, subcutaneous tumor volume, organ weights and RNA procedures of liver, spleen, kidney, and ovaries.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. An isolated or synthetic oligonucleotide inhibitor of DNA methyltransferase comprising at least one CpG site, wherein the oligonucleotide inhibitor is a self-complementary single stranded oligonucleotide sequence in the form of a stem-loop structure at human body temperature, and wherein the cytosine of the CpG site is a cytosine analog selected from the group consisting of:
a. zebularine; and
b. deoxyzebularine.

2. The isolated or synthetic oligonucleotide inhibitor of DNA methyltransferase of claim 1, wherein the nucleic acid sequence of the oligonucleotide is selected from the group consisting of:
a. an isolated or synthetic oligonucleotide comprising or consisting of the nucleotide sequence of SEQ ID NO:1;
b. an isolated or synthetic oligonucleotide comprising or consisting of the nucleotide sequence of SEQ ID NO:2;
c. an isolated or synthetic oligonucleotide comprising or consisting of the nucleotide sequence of SEQ ID NO:4;
d. an isolated or synthetic oligonucleotide comprising or consisting of the nucleotide sequence of SEQ ID NO:6;
e. an isolated or synthetic oligonucleotide comprising or consisting of the nucleotide sequence of SEQ ID NO:11;
f. an isolated or synthetic oligonucleotide comprising or consisting of the nucleotide sequence of SEQ ID NO:12;
g. an isolated or synthetic oligonucleotide comprising or consisting of the nucleotide sequence of SEQ ID NO:14;
h. an isolated or synthetic oligonucleotide comprising or consisting of the nucleotide sequence of SEQ ID NO:16;
i. an isolated or synthetic oligonucleotide comprising or consisting of the nucleotide sequence of SEQ ID NO:18;
j. an isolated or synthetic oligonucleotide comprising or consisting of the nucleotide sequence of SEQ ID NO:19;
k. an isolated or synthetic oligonucleotide comprising or consisting of the nucleotide sequence of SEQ ID NO:25;
l. an isolated or synthetic oligonucleotide comprising or consisting of the nucleotide sequence of SEQ ID NO:26;
m. an isolated or synthetic oligonucleotide comprising or consisting of the nucleotide sequence of GENERAL FORMULA A
n. an isolated or synthetic oligonucleotide comprising or consisting of the nucleotide sequence of GENERAL FORMULA B;
o. an isolated or synthetic oligonucleotide comprising or consisting of the nucleotide sequence of GENERAL FORMULA C;
p. an isolated or synthetic oligonucleotide comprising or consisting of the nucleotide sequence of GENERAL FORMULA D; and
q. any combination of two or more of a)-p).

3. The isolated or synthetic oligonucleotide inhibitor of DNA methyltransferase of
claim 1, wherein the cytosine of the sequence that is complementary to the guanine of the CpG site is 5-methylcytosine.

4. The isolated or synthetic oligonucleotide inhibitor of DNA methyltransferase of claim 1, wherein the backbone linker of the oligonucleotide inhibitor is an artificial backbone.

5. The isolated or synthetic oligonucleotide inhibitor of DNA methyltransferase of claim 5, wherein the backbone linker is resistant to nuclease degradation in vivo.

6. The isolated or synthetic oligonucleotide inhibitor of DNA methyltransferase of claim 1, wherein the oligonucleotide inhibitor is provided in a composition.

7. The composition of claim 6, wherein the composition is a pharmaceutical composition.

8. A method for reducing, limiting, inhibiting, or minimizing methylation of a cell, comprising contacting the cell under suitable conditions with an agent that comprises a composition comprising the isolated or synthetic oligonucleotide inhibitor of DNA methyltransferase of claim 1, or a pharmaceutically acceptable salt or ester thereof, and whereby methylation in the cell is reduced, limited, inhibited, or minimized.

9. The method of claim 8, wherein the methylation is measured by a reduction, inhibition, or minimization, of methylation in genomic DNA.

10. A method of treating a DNA Methyltransferase (DNMT) related disease or disorder in a subject in need thereof, the method comprising administering to the subject a therapeutically effective amount of a composition comprising the isolated or synthetic oligonucleotide inhibitor of DNA methyltransferase of claim 1.

11. The method of claim 10, wherein said DNMT related disease or disorder is a cell proliferative disorder.

12. The method of claim 11, wherein said cell proliferative disorder is selected from the group consisting of:
a. acute-myeloid leukemia (AML);
b. chronic myeloid leukemia (CML);
c. myelodysplastic syndromes (MDS);
d. liver cancer or liver proliferative disorder;
e. kidney cancer or a kidney proliferative disorder;
f. ovarian cancer or ovarian proliferative disorder
g. breast cancer or breast proliferative disorder;
h. colorectal cancer or colorectal proliferative disorder;
i. lung cancer or lung proliferative disorder; and
j. pancreatic cancer or pancreatic proliferative disorder.

13. The isolated or synthetic oligonucleotide inhibitor of claim 1, wherein the nucleic acid sequence of the oligonucleotide is selected from the group consisting of:
a. an isolated or synthetic oligonucleotide comprising or consisting of the nucleotide sequence of SEQ ID NO:1;
b. an isolated or synthetic oligonucleotide comprising or consisting of the nucleotide sequence of SEQ ID NO:2;
c. an isolated or synthetic oligonucleotide comprising or consisting of the nucleotide sequence of SEQ ID NO:3 (MTC-422);
d. an isolated or synthetic oligonucleotide comprising or consisting of the nucleotide sequence of SEQ ID NO:4 (MTC-423);
e. an isolated or synthetic oligonucleotide comprising or consisting of the nucleotide sequence of SEQ ID NO:5 (MTC-425);
f. an isolated or synthetic oligonucleotide comprising or consisting of the nucleotide sequence of SEQ ID NO:6 (MTC-427);
g. an isolated or synthetic oligonucleotide comprising or consisting of the nucleotide sequence of SEQ ID NO:7 (MTC-424F);
h. an isolated or synthetic oligonucleotide comprising or consisting of the nucleotide sequence of SEQ ID NO:8 (MTC-424R);
i. an isolated or synthetic oligonucleotide comprising or consisting of the nucleotide sequence of SEQ ID NO:9 (MTC-429F);
j. an isolated or synthetic oligonucleotide comprising or consisting of the nucleotide sequence of SEQ ID NO:10 (MTC-429R);
k. an isolated or synthetic oligonucleotide comprising or consisting of the nucleotide sequence of SEQ ID NO:11 (MTC-432);
l. an isolated or synthetic oligonucleotide comprising or consisting of the nucleotide sequence of SEQ ID NO:12 (MTC-433);
m. an isolated or synthetic oligonucleotide comprising or consisting of the nucleotide sequence of SEQ ID NO:13 (MTC-434);
n. an isolated or synthetic oligonucleotide comprising or consisting of the nucleotide sequence of SEQ ID NO:14 (MTC-443);
o. an isolated or synthetic oligonucleotide comprising or consisting of the nucleotide sequence of SEQ ID NO:15 (MTC-422N);
p. an isolated or synthetic oligonucleotide comprising or consisting of the nucleotide sequence of SEQ ID NO:16 (MTC-423N);
q. an isolated or synthetic oligonucleotide comprising or consisting of the nucleotide sequence of SEQ ID NO:17 (MTC-425N);
r. an isolated or synthetic oligonucleotide comprising or consisting of the nucleotide sequence of SEQ ID NO:18 (MTC-427N);
s. an isolated or synthetic oligonucleotide comprising or consisting of the nucleotide sequence of SEQ ID NO:19 (MTC-432N);
t. an isolated or synthetic oligonucleotide comprising or consisting of the nucleotide sequence of SEQ ID NO:20 (MTC-433N);
u. an isolated or synthetic oligonucleotide comprising or consisting of the nucleotide sequence of SEQ ID NO:21 (MTC-424FN);
v. an isolated or synthetic oligonucleotide comprising or consisting of the nucleotide sequence of SEQ ID NO:22 (MTC-424RN);
w. an isolated or synthetic oligonucleotide comprising or consisting of the nucleotide sequence of SEQ ID NO:23 (MTC-429FN);
x. an isolated or synthetic oligonucleotide comprising or consisting of the nucleotide sequence of SEQ ID NO:24 (MTC-429RN);
y. an isolated or synthetic oligonucleotide comprising or consisting of the nucleotide sequence of GENERAL FORMULA A;
z. an isolated or synthetic oligonucleotide comprising or consisting of the nucleotide sequence of GENERAL FORMULA B;
aa. an isolated or synthetic oligonucleotide comprising or consisting of the nucleotide sequence of GENERAL FORMULA C;
bb. an isolated or synthetic oligonucleotide comprising or consisting of the nucleotide sequence of GENERAL FORMULA D;
cc. an isolated or synthetic pair of oligonucleotides comprising or consisting of the nucleotide sequence of SEQ ID NO:7, and its complementary sequence;
dd. an isolated or synthetic pair of oligonucleotides comprising or consisting of the nucleotide sequence of SEQ ID NO:8, and its complementary sequence;
ee. an isolated or synthetic pair of oligonucleotides comprising or consisting of the nucleotide sequence of SEQ ID NO:7 and the nucleotide sequence of SEQ ID NO:8; and
ff. any combination of two or more of a)-ff).

14. The isolated or synthetic oligonucleotide inhibitor of DNA methyltransferase of claim 13, wherein the cytosine of the sequence that is complementary to the guanine of the CpG site is 5-methylcytosine.

15. The isolated or synthetic oligonucleotide inhibitor of DNA methyltransferase of claim 13, wherein the backbone linker of the oligonucleotide inhibitor is an artificial backbone.

16.-21. (canceled)

22. The method of claim 8, wherein the method comprises reverting aberrant methylation of a cell, whereby aberrant methylation in the cell is reverted in whole or in part.

23. The method of claim 8, wherein the method comprises restoring hypo-methylation of a tumor suppressor gene whereby the tumor suppressor gene is hypo-methylated in whole or in part.

24. The method of claim 8, wherein the method comprises restoring transcriptional activity of a tumor suppressor gene whereby transcriptional activity of tumor suppressor genes is restored whole or in part.

25. The method of claim 8, wherein the method comprises introducing re-expression of a methylation-silenced tumor suppressor gene whereby re-expression of methylation-silenced tumor suppressor genes is restored in whole or in part.

26. The method of claim 8, wherein the method comprises inhibiting, reducing, limiting, or minimizing tumorgenecity of a gene whereby tumorgenecity of the gene is inhibited, reduced, limited, or minimized in whole or in part.

27. (canceled)
